(19) United States
(12) Patent Application Publication
(10) Pub. No.: US 2012/0064136 A1
(43) Pub. Date: Mar. 15, 2012

(54) ANTI-AGING AND WRINKLE TREATMENT METHODS USING NANOEMULSION COMPOSITIONS

(75) Inventors: James R. Baker, JR., Ann Arbor, MI (US); Susan M. Ciotti, Ann Arbor, MI (US)

(73) Assignee: NanoBio Corporation

(21) Appl. No.: 13/229,080

(22) Filed: Sep. 9, 2011

Related U.S. Application Data
(60) Provisional application No. 61/381,833, filed on Sep. 10, 2010.

(51) Int. Cl. A61K 8/06; A61K 8/92; A61K 31/192; A61K 31/327; A61K 31/60; A61Q 17/04; A61Q 19/08; A61Q 19/00; A61Q 19/02; A61P 17/00; A61P 17/10; A61P 31/12; A61P 17/02; A61K 9/107; B82Y 5/00

(52) U.S. Cl. 424/401; 424/400; 424/59; 424/62; 514/569; 514/714; 514/159; 977/788; 977/906; 977/926

(57) ABSTRACT

The present invention relates to methods for treating, preventing, minimizing, and/or diminishing signs of aging in the skin comprising administering to the subject in need thereof a nanoemulsion composition.

### CROSS-REFERENCE TO RELATED APPLICATION

This application claims benefit of U.S. provisional Application No. 61/381,833, filed on Sep. 10, 2010, the disclosure of which is incorporated herein by reference.

### FIELD OF THE INVENTION

The present disclosure relates to methods for preventing and/or treating wrinkles and signs of aging skin. The method comprises topically or subcutaneously administering to a subject in need thereof a nanoemulsion composition having anti-wrinkle and anti-aging properties.

### BACKGROUND OF THE INVENTION

### A. Background Regarding Wrinkles and Aging Skin

Wrinkles are a natural part of aging. As a person ages, skin gets thinner, drier and less elastic. And it becomes less able to protect itself from damage. As a result, wrinkles, lines and creases form in the skin. Some wrinkles can become deep crevices or furrows and may be especially noticeable around the eyes, mouth and neck. McCullough J L, et al., “Prevention and treatment of skin aging,” Annals of the New York Academy of Sciences, 1067:323 (2006). Other signs of aging skin include, but are not limited to, liver spots or age spots (solar lentigines), uneven skin tone, sun-damaged skin, and acne or chickenpox scars.

Wrinkles are caused by a combination of factors, such as age, exposure to ultraviolet (UV) light, smoking, and repeated facial expressions. Decreased production of natural oils makes skin drier and appear more wrinkled. Fat in the deeper layers of skin, which gives the skin a plump appearance, starts to lessen. This causes loose, saggy skin and more pronounced lines and crevices. Ultraviolet radiation markedly speeds up the natural aging process and is the primary cause of early wrinkling Exposure to UV light breaks down the skin's connective tissue—collagen and elastin fibers, which lie in the deeper layer of skin (dermis). Without the supportive connective tissue, skin loses its strength and flexibility. As a result, skin begins to sag and wrinkle prematurely. Smoking can accelerate the normal aging process of skin, contributing to wrinkles. This may be due to changes in the blood supply to the skin. Finally, facial movements and expressions, such as squinting or smiling, lead to fine lines and wrinkles. Each time a facial muscle is used, a groove forms beneath the surface of the skin. And as skin ages, it loses its flexibility and is no longer able to spring back in place. These grooves then become permanent features on the face. See Helfrich et al., “Overview of skin aging and photoaging,” Dermatology Nursing, 20:177 (2008); Freiman et al., “Cutaneous effects of smoking,” Journal of Cutaneous Medicine and Surgery, 8:415 (2004); Just et al., “Effect of smoking on skin elastic fibres: Morphometric and immunohistochemical analysis,” British Journal of Dermatology, 156:85 (2007).

### B. Conventional Treatment for Wrinkles and Aging Skin

Current wrinkle and anti-aging treatments for skin include medications and surgical procedures and other techniques. However, all of the known treatments have disadvantages and undesirable side effects.

1. Medications and Cosmetics

Commercially available medications used to treat wrinkles and signs of skin aging include topical retinoids and non-prescription wrinkle creams. Topical retinoids are derived from vitamin A, are applied to the skin, and may be able to reduce fine wrinkles, splotchy pigmentation and skin roughness. Retinoids must be used with a skin-care program that includes sunscreen and protective clothing because the medication can make skin burn more easily. Retinoids may also cause redness, dryness, itching, and a burning or tingling sensation. Tretinoin (Renova®, Retin-A®) and tazarotene (Avage®, Tazorac®) are examples of topical retinoids. See Renova (tretinoin cream). U.S. Food and Drug Administration. http://www.fda.gov/ohrms/dockets/ac/02/sides/3904S1_{—}01_Bergfeld/sld024.htm (accessed Jun. 22, 2010); Kafi et al., “Improvement of naturally aged skin with vitamin A (retinol),” Archives of Dermatology, 143:606 (2007).

The effectiveness of non-prescription anti-wrinkle creams depends in part on the active ingredient or ingredients. Retinol, alpha hydroxy acids, kinetin, coenzyme Q10, copper peptides, and antioxidants may result in slight to modest improvements in wrinkles and signs of aging skin. Baumann L., “Cosmetics and skin care in dermatology,” In: Wolff et al., Fitzpatrick's Dermatology in General Medicine, 7th ed. (New York, N.Y.: The McGraw-Hill Companies; 2008). However, nonprescription wrinkle creams contain lower concentrations of active ingredients (such as alpha hydroxy acids) and other structurally different actives (such as retinoids) than do prescription creams. Therefore the claims and efficacy, if any, are limited and usually short-lived.

Many wrinkle creams and lotions sold in department stores, in drugstores and on the Internet promise to reduce wrinkles and prevent or reverse damage caused by the sun. Many of these products are not likely to make a noticeable difference in the skin. The Food and Drug Administration (FDA) classifies these creams and lotions as cosmetics, which are defined as having no medical value. The FDA regulates cosmetic products less strictly than it does drugs products. This means that products do not need to undergo rigorous testing for safety and effectiveness before approval to go on the market. The FDA does not evaluate cosmetic products for medical effectiveness, and there is no guarantee that any over-the-counter product will reduce wrinkles. The claims for cosmetic products are cosmetic “appearance” claims and are not medical claims. However, it is difficult for the consumer to delineate between the two types of claims. For example, a drug claim for wrinkles can be “reduces fine line and wrinkles”. An acceptable cosmetic claim for photoaging can be: “visibly reduces the appearance of fine fines and deep wrinkles caused by photoaging or sun damage”. Including the words “visibly” and “appearance” converts the drug claim to a cosmetic claim. This small change is not completely understood by the consumer.

The effectiveness of anti-wrinkle creams depends in part on the active ingredient or ingredients. Examples of some common ingredients that may result in slight to modest improvements in wrinkles include retinol, hydroxyl acids, Coenzyme Q10, copper peptides, Kinetin, and tea extracts.

Retinol is a vitamin A compound and is the first antioxidant to be widely used in nonprescription wrinkle creams. Antioxidants are substances that neutralize free radicals unstable oxygen molecules that break down skin cells and cause wrinkles Retinol is less potent than the vitamin A derivative tretinoin, a topical treatment approved by the Food and Drug Administration (FDA) for treating wrinkles Tretinoin is available only by prescription. Vitamin A derivatives are counter-indicated for pregnant women as they increase the risk of birth defects.

Alpha hydroxy acids, beta hydroxy acids and poly hydroxy acids are all synthetic versions of acids derived from sugar-containing fruits (“hydroxyl acids”). These acids are exfoliants—substances that remove the upper layer of old, dead skin and stimulate the growth of smooth, evenly pigmented new skin.

Other examples of cosmetic ingredients that may be effective in treating wrinkles include Coenzyme Q10, copper peptides, kinetin, and tea extracts. Coenzyme Q10 is a nutrient that helps regulate energy production in cells. Some studies have shown reduction in fine wrinkles around the eyes with no side effects. Other studies show that application before sun exposure protects against sun damage. Copper is a trace element found in every cell. In products applied to the skin, it's combined with small protein fragments called peptides.

Copper peptides enhance wound healing. Copper peptides also stimulate production of collagen and may enhance the action of antioxidants. A plant growth factor, kinetin may improve wrinkles and uneven pigmentation with minimal irritation. It's unclear how it works, but it may help reduce wrinkles by helping skin retain moisture and by stimulating the production of collagen. Kinetin may also be a potent antioxidant. Finally, green, black and oolong tea contain compounds with antioxidant and anti-inflammatory properties. Green tea extracts are most commonly found in wrinkle creams. Side effects of such cosmetic ingredients include skin irritation, rashes, and burning or redness.

2. Surgical Procedures and Other Techniques

Surgical procedures and other techniques used to treat wrinkles and signs of aging skin include a variety of skin-resurfacing techniques, injectables, fillers and surgical procedures to smooth out wrinkles and treat signs of aging skin. Each works a little differently and has its own set of potential results and side effects. Examples include dermabrasion, microdermabrasion, laser, chemical peel, Botulinum toxin type A (Botox), soft tissue fillers, face lift, and other surgical techniques.

Dermabrasion consists of sanding down (planing) the surface layer of skin with a rapidly rotating brush. The planing removes the skin surface and a new layer of skin grows in its place. Redness, scabbing and swelling generally last a couple of weeks. It may take several months for pinkness to fade and to see the desired results. Microdermabrasion is similar to dermabrasion, but less surface skin is removed. It's done using a vacuum suction over the face while aluminum oxide crystals essentially sandblast the skin. Only a fine layer of skin is removed. A slight redness to the treated areas may occur. Microdermabrasion usually requires repeated treatments to maintain the subtle, temporary results.

Laser resurfacing removes aged or sun-damaged skin to allow younger looking skin to grow in its place. Laser resurfacing is an effective treatment for minor facial flaws. For example, it can lessen the appearance of fine lines around the eyes, mouth and cheeks. It can also improve a complexion having yellowish or grayish skin tones. In ablative (wounding) laser resurfacing, a laser beam destroys the outer layer of skin (epidermis) and heats the underlying skin (dermis), which stimulates the growth of new collagen fibers. As the wound heals, new skin forms that's smoother and tighter. It can take up to several months to fully heal from ablative laser resurfacing. Newer developments in laser technology have decreased the healing time. Less intense lasers (nonablative lasers), pulsed light sources and radiofrequency devices don't injure the epidermis. These treatments heat the dermis and cause new collagen and elastin formation. After several treatments, skin feels firmer and appears refreshed. This means shorter recovery times, but treatment typically needs to be repeated more often and results are subtle. Laser resurfacing can treat fine to moderate wrinkles, liver spots or age spots (solar lentigines), uneven skin tone, sun-damaged skin, and acne or chickenpox scars. Laser resurfacing has limitations. It can't remove deep wrinkles or eliminate excessive or sagging skin (jowls). In addition, the effects aren't permanent because as a person ages, they continue to acquire expression lines—lines that result from the natural movement of the face, such as a person squints or smiles. Complications of ablative laser resurfacing can include hyperpigmentation or hypopigmentation (skin tone that turns darker or lighter than normal), herpes virus infection (the virus that causes cold sores), bacterial infection, acne flares, small white bumps (milia), scarring, burns, inflammation of the skin (dermatitis), and prolonged redness. For a herpes virus infection, the herpes virus is already present but dormant in the skin; laser resurfacing can cause the virus to flare up.

For a chemical peel, an acid is applied to the skin, which burns the outer layer of the skin. With medium-depth peels, the entire epidermis and a small portion of the dermis are removed. New skin forms to take its place. The new skin is usually smoother and less wrinkled than the old skin. Redness lasts up to several weeks. With superficial peels, only a portion of the epidermis is removed. After a series of peels, less fine wrinkling in the skin and a fading of brown spots may be observed.

Botulinum toxin type A (Botox®), when injected in small doses into specific muscles, blocks the chemical signals that cause muscles to contract. When the muscles can't tighten, the skin flattens and appears smoother and less wrinkled. Botox® works well on frown lines between the eyebrows and on the bridge of the nose, forehead creases, crow's-feet at the corners of the eyes, and skin bands on the neck. Results typically last about three to four months. Repeat injections are needed to maintain results. Not all facial wrinkles benefit from Botox® injections, however. Botox® won't reverse wrinkling caused by sun damage. Also, it's less desirable to treat the lines around the mouth because muscles in this area are needed for eating and talking Side effects and complications of Botox® injections include pain and bruising at the injection site, redness, headache, flu-like symptoms, nausea, temporary facial weakness or drooping, and spread of the toxin beyond the treatment area, which can cause botulism-like signs and symptoms (trouble swallowing, muscle weakness, slurred speech and breathing problems).

Soft tissue fillers, which include fat, collagen and hyaluronic acid (Restylane®, Juvederm®), can be injected into deeper wrinkles on the face. They plump and smooth out wrinkles and furrows and give the skin more volume. Side effects include temporary swelling, redness and bruising in the treated area. The procedure may need to be repeated every few months.

A face-lift procedure involves removing excess skin and fat in the lower face and neck and tightening the underlying muscle and connective tissue. The results typically last five to 10 years. Healing times can be lengthy after a face-lift. Bruising and swelling are usually evident for several weeks after surgery.

### C. Background Regarding Nanoemulsions

Prior teachings related to nanoemulsions are described in U.S. Pat. No. 6,015,832, which is directed to methods of inactivating a Gram-positive bacteria, a bacterial spore, or a Gram-negative bacteria. The methods comprise contacting the Gram-positive bacteria, bacterial spore, or Gram-negative bacteria with a bacteria-inactivating (or bacterial-spore inactivating) emulsion. U.S. Pat. No. 6,506,803 is directed to methods of killing or neutralizing microbial agents (e.g., bacteria, virus, spores, fungus, on or in humans using an emulsion. U.S. Pat. No. 6,559,189 is directed to methods for decontaminating a sample (human, animal, food, medical device, etc.) comprising contacting the sample with a nanoemulsion. The nanoemulsion, when contacted with bacterial, virus, fungi, protozoa, or spores, kills or disables the pathogens. The antimicrobial nanoemulsion comprises an oil, quaternary ammonium compound, one of ethanol/glycerol/PEG, a surfactant, and water. U.S. Pat. No. 6,635,676 is directed to two different compositions and methods of decontaminating samples by treating a sample with either of the compositions. Composition 1 comprises an emulsion that is antimicrobial against bacteria, virus, fungi, protozoa, and/or spores. The emulsions comprise an oil and a quaternary ammonium compound. U.S. Pat. No. 7,314,624 is directed to methods of inducing an immune response to an immunogen comprising treating a subject via a mucosal surface with a combination of an immunogen and a nanoemulsion. The nanoemulsion comprises oil, ethanol, a surfactant, a quaternary ammonium compound, and distilled water. US-2005-0208083-A1 and US-2006-0251684-A1 are directed to nanoemulsions having droplets with preferred sizes. US-2007-0054834-A1 is directed to compositions comprising quaternary ammonium halides and methods of using the same to treat infectious conditions. The quaternary ammonium compound may be provided as part of an emulsion. Finally, US-2007-0036831-A1 is directed to nanoemulsions comprising an anti-inflammatory agent.

Examples of documents describing compositions for treating wrinkles or skin aging include U.S. Pat. No. 6,896,889 for “Immediate effect anti-wrinkle composition, based on an aqueous dispersion, of at least one mineral filler,” directed to a composition comprising colloid particles of a mineral filler. U.S. Pat. No. 6,808,715 for “Wrinkle Cream,” is directed to an emulsion comprising water, hydrophilic particles, and hydrophobic particles, wherein the hydrophilic and hydrophobic particles form shells encapsulating a gas that is suspended in the water. U.S. Pat. No. 6,497,890 for “Anti-wrinkle preparation and method of reducing wrinkles in facial skin and neck,” is directed to a method for the prevention or minimization of wrinkles in the face and neck areas of a patient by topically applying finely divided safflower seeds or extract thereof in combination with a pharmaceutically acceptable carrier. U.S. Pat. No. 6,344,188 for “Wrinkle reducing cream,” is directed to a cream comprising water, caffeine, and glycerin. U.S. Pat. No. 5,360,824 for “Human skin cleansing and wrinkle-reducing cream,” is directed to a composition comprising water-soluble granules which can be an inorganic salt, such as a water-soluble vitamin and/or water-soluble vitamin-yielding salt, an oil and a petrolatum jelly. U.S. Pat. No. 4,777,041 for “Wrinkle treatment formulation,” is directed to compositions comprising a gelable hydrophilic polyurethane polymer base and a precipitated silica thickener gelling agent. U.S. Pat. No. 7,384,916 for “Methods and compositions for preventing and treating aging or photodamaged skin,” is directed to topical compositions comprising a peptide manganese complex. U.S. Pat. Nos. 7,354,610 and 7,214,395, both for “Pharmaceutical and cosmetic composition against skin aging,” is directed to compositions comprising phospholipid complexes of extracts of Vitis vinifera, and phospholipid complexes of standardized extract from Centella asiatica. U.S. Pat. No. 7,205,003 for “Method and topical formulation for treating skin conditions associated with aging,” is directed to topical compositions comprising a cosmeceutically active base, which is either an inorganic base, such as an inorganic hydroxide, an inorganic oxide, or a metal salt of a weak acid, or an organic base, such as a nitrogenous base.

The intact skin of humans is an effective barrier to many natural and synthetic substances. Many cosmetic and pharmaceutical agents, which are pharmacologically effective on oral or systemic administration, may be much less effective or even totally ineffective, when applied topically to the skin. Therefore, there is an ongoing need in the art for new and effective regimens for treating aging-related skin conditions. The present invention addresses these and other needs in the art by providing novel methods and topical formulations for treating a variety of aging-related skin conditions, including wrinkles, age spots, sun damage (particularly UV radiation-induced oxidative stress/photodamage), blemishes, hyperpigmented skin, age spots, increased skin thickness, loss of skin elasticity and collagen content, dry skin, lentigines, and melasmas.

There remains a need for more effective and otherwise improved methods for treating dermatological conditions related to aging skin, such as fine lines and wrinkles, and skin imperfections, such as scars. The present invention addresses these needs and provides further related advantages.

### SUMMARY OF THE INVENTION

The present invention provides methods for treating, preventing, and/or minimizing wrinkles, signs of aging skin, and/or skin imperfections comprising administering a nanoemulsion to a subject. Examples of signs of aging skin and/or skin imperfections which can be treated, prevented, and/or minimized with the methods of the invention include, but are not limited to, (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content (laxity and firmness), (10) melasmas (a typical pigmentation or hyper-pigmentation of the skin), (11) skin clarity and/or radiance, (12) skin smoothness and/or softness, (13) pore size (larger pore can make an individual appear older), (14) increase hydration, (15) increase skin smoothness, (16) increase skin tightness, and any combination thereof.

The compositions of the invention can also be used to treat scars, such as acne and chickenpox scars. Collectively the signs of aging skin, skin imperfections and scars are referred to as “dermatological conditions.” The nanoemulsion comprises droplets having an average diameter of less than about 3 microns, and the nanoemulsion droplets comprise an aqueous phase, at least one oil, at least one surfactant, and at least one organic solvent.

In one embodiment the nanoemulsion is applied topically, which is a non-invasive administration technique. In an alternative embodiment, the nanoemulsion can be applied subcutaneously.

In an exemplary embodiment, a method of the invention for treating, reducing and/or minimizing the dermatological conditions described above (e.g., wrinkling, signs of aging skin, and/or skin imperfections) in a region of skin comprises applying a nanoemulsion according to the invention to the region of skin. The nanoemulsion can be applied to any skin region of a subject. In one embodiment, the nanoemulsion is applied to the facial tissue of a subject. In another embodiment, the nanoemulsion is applied to the neck tissue of a subject. It has been surprisingly found that the nanoemulsion compositions of the invention can be used to substantially treat, reduce, minimize, and/or diminish the dermatological conditions described above.

In a further embodiment, the nanoemulsion composition of the invention further comprises one or more active agents. The presence of such an active agent is not required, and substantial and unexpected anti-aging and/or anti-wrinkle properties are observed without the presence of such an active agent. Thus, the presence of such an active agent is to merely enhance the anti-aging and/or anti-wrinkle properties of the compositions of the invention. In one embodiment, an active agent incorporated into a nanoemulsion composition of the invention is a compound that provide benefits to the skin and/or provides desirable properties to a composition formulated as a cosmetic or medicinal preparation. The active agent can be a drug substance or a non-drug substance. Examples of non-drug active agents include, but are not limited to, skin lightening agents, tanning agents, skin conditioning agents, skin protectants, emollients and humectants.

Examples of exemplary active agents, such as an active drug substance or an active cosmetic substance, that can be incorporated into a nanoemulsion composition of the invention include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac®), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids) or any mixture thereof.

In another embodiment, the composition of the invention comprises an anti-viral agent. Such a composition is useful, for example, in treating a herpes virus outbreak following laser treatment, such as ablative laser resurfacing. In yet another embodiment, the composition of the invention comprises an anti-acne agent.

The composition of the invention allows for targeted delivery to the area of skin to be treated. Moreover, the nanoemulsions are able to enhance delivery, and thus effectiveness, of other active drug agents and/or active cosmetic agents incorporated into the nanoemulsion, thereby enhancing the efficacy and reducing the detrimental side effects of the other agents.

In certain embodiments of the invention, the nanoemulsion can have an increased viscosity to aid in permeation of the nanoemulsion into the dermis and epidermis.

In other embodiments of the invention, the nanoemulsion at the time of topical application is at room temperature or warmer.

The nanoemulsion comprises droplets having an average particle size of less than about 3 microns, and the nanoemulsion comprises water, at least one oil, at least one surfactant, and at least one organic solvent. In one embodiment of the invention, the surfactant present in the nanoemulsion is a cationic surfactant. In another embodiment of the invention, the nanoemulsion further comprises a chelating agent. In one embodiment of the invention, nanoemulsions from the present invention, or those derived from the nanoemulsions of the present invention, are diluted. The diluted samples can then be tested to determine if they maintain the desired functionality, such as surfactant concentration, stability, and particle size.

Preferably, the nanoemulsions are in the form of any pharmaceutically acceptable dosage form, including but not limited to, ointments, creams, emulsions, lotions, gels, liquids, bioadhesive gels, sprays, shampoos, aerosols, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within the epidermis, the dermis and keratin layers. Preferably, the nanoemulsions are in a dosage form suitable for topical administration. The nanoemulsion is capable of effectively treating and/or preventing signs of aging associated with the skin, such as fine to moderate wrinkles, uneven skin tone, and other dermatological signs of aging skin noted above, without being systemically absorbed and without significantly irritating the skin.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the cross-section view of the pilosebaceous unit in human cadaver skin and hamster ear after application of nanoemulsion plus fluorescein.

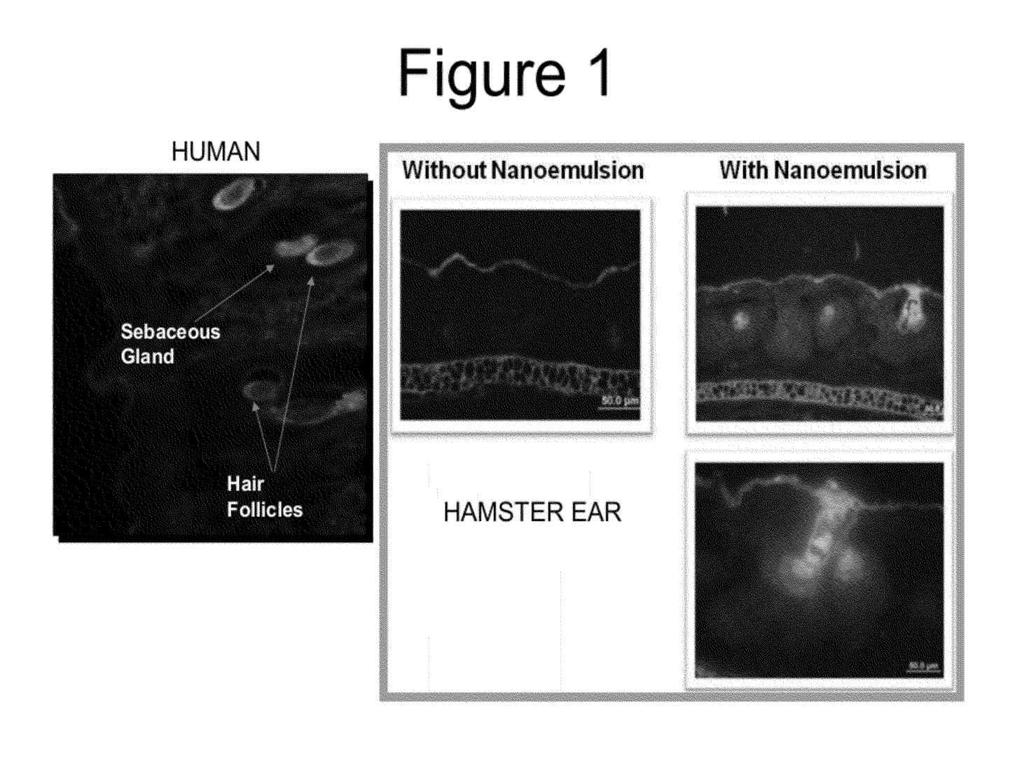

FIG. 2 shows in vitro skin permeation of nanoemulsion formulations into the epidermal layer of pig abdominal skin at 24 hours after a single topical application of 100 μl/cm².

FIG. 3 shows in vitro permeation of nanoemulsion formulations in pig abdominal skin (epidermis and dermis) at 12 and 24 hours after a single topical application of 100 μl/cm².

FIG. 4 shows the effect the concentration of a nanoemulsion has on the particle size and viscosity of the nanoemulsion. With a decrease in concentration of the active, viscosity (cP) declines (triangles), whereas the particle size remains constant (bars).

FIG. 5 shows the results of a permeation study utilizing pig skin epidermis with 5 skin sections (n=5) following administration of a nanoemulsion (NB-003) twice daily (BID). Higher viscosity (greater than 1000 cps) nanoemulsions (e.g., 0.8% NB-003) were found to enhance permeation of the nanoemulsion into the epidermis.

FIG. 6 shows the results of a permeation study utilizing pig skin dermis with 5 skin sections (n=5) following administration of a nanoemulsion (NB-003) twice daily (BID). Higher viscosity (greater than 1000 cps) nanoemulsions (e.g., 0.8% NB-003) were found to deliver three times the amount of the surfactant, cetylpyridinium chloride (CPC) to the dermis as compared to a lower viscosity nanoemulsion (e.g., 0.25% NB-003).

FIG. 7 shows the effect of storage temperature of a nanoemulsion (e.g., NB-003) on the in vitro activity of the nanoemulsion against P. acnes in the presence of sebum.

FIG. 8 shows the results of the permeation study utilizing pig skin following administration of a nanoemulsion formulated with benzoyl peroxide twice daily (BID) as compared to a commercial preparation of 10% benzoyl peroxide.

FIG. 9 shows the results of the permeation study utilizing pig skin following topical administration (100 μl/cm²) of a nanoemulsion with incorporated 0.1% adapalene twice daily (BID) as compared to two different commercial preparations of adapalene.

FIG. 10 shows the results of the permeation study utilizing female cadaver abdominal skin following topical administration (100 μl/cm²) of a nanoemulsion and incorporated 0.1% adapalene once daily (QD) as compared to two different commercial preparations of adapalene.

### DETAILED DESCRIPTION OF THE PREFERRED EMBODIMENTS

The present invention provides methods for treating, preventing, and/or minimizing wrinkles, signs of aging skin, and/or skin imperfections comprising administering a nanoemulsion to a subject. Examples of signs of aging skin and/or skin imperfections which can be treated, prevented, and/or minimized with the methods of the invention include, but are not limited to, (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content, (10) melasmas (atypical pigmentation or hyper-pigmentation of the skin), (11) skin clarity and/or radiance, (12) skin smoothness and/or softness, (13) pore size (larger pore can make an individual appear older), (14) increase hydration, (15) increase skin smoothness, (16) increase skin tightness, and any combination thereof. The compositions of the invention can also be used to treat scars, such as acne and chickenpox scars. Collectively the signs of aging skin, skin imperfections and scars are referred to as “dermatological conditions.”

As described in the examples below, following topical application of a nanoemulsion to facial skin, up to 75% of nanoemulsion users reported that skin was smoother and softer. Moreover, up to 37.5% of nanoemulsion users reported that following treatment skin demonstrated more clarity and/or radiance, and up to 37.5% of nanoemulsion users reported that wrinkles appeared softer and/or less prominent. In addition, following treatment up to 35% of nanoemulsion users reported that the overall appearance of skin was improved, up to 30% of nanoemulsion users reported that skin tone and skin texture was more even, and up to 25% of nanoemulsion users reported that following treatment skin looked and/or felt younger.

In an exemplary embodiment, a method of the invention for treating, reducing and/or minimizing dermatological conditions in a region of skin comprises applying a nanoemulsion according to the invention to the region of skin. In one embodiment the nanoemulsion is applied topically, which is a non-invasive administration technique. In an alternative embodiment, the nanoemulsion can be applied subcutaneously. The nanoemulsion can be applied to any skin region of a subject. In one embodiment, the nanoemulsion is applied to the facial tissue of a subject. In another embodiment, the nanoemulsion is applied to the neck tissue of a subject. It has been surprisingly found that the nanoemulsion compositions of the invention can be used to substantially treat, minimize, and/or diminish the dermatological conditions described above.

In a further embodiment, the composition additionally comprises one or more active agents. The presence of such an active agent is not required, and substantial and unexpected anti-aging and/or anti-wrinkle properties are observed without the presence of such an active agent. Thus, the presence of such active agents is to merely enhance the anti-aging and anti-wrinkle properties of the compositions of the invention. In one embodiment, active agents useful in the nanoemulsion compositions of the invention are compounds that provide benefits to the skin and/or provide desirable properties to a composition formulated as a cosmetic or medicinal preparation. The active agent useful in the nanoemulsion compositions of the invention can be a drug substance or a non-drug substance. Examples of non-drug active agents include, but are not limited to, skin lightening agents, tanning agents, skin conditioning agents, skin protectants, emollients and humectants.

Examples of exemplary active agents useful in the nanoemulsion compositions of the invention, such as an active drug substance or an active cosmetic substance, include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac®), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids) or any mixture thereof.

In another embodiment, the composition of the invention comprises an anti-viral agent. Such a composition is useful, for example, in treating a herpes virus outbreak following laser treatment, such as ablative laser resurfacing. In yet another embodiment, the composition of the invention comprises an anti-acne agent.

The nanoemulsion composition of the invention allows for targeted delivery to the area of skin to be treated. Moreover, the nanoemulsions are able to enhance delivery, and thus effectiveness, of other active drug agents and/or active cosmetic agents incorporated into the nanoemulsion, thereby enhancing the efficacy and reducing the detrimental side effects of the other agents.

In certain embodiments of the invention, the nanoemulsion can have an increased viscosity to aid in permeation of the nanoemulsion into the dermis and epidermis. In other embodiments of the invention, the nanoemulsion at the time of topical application is at room temperature or warmer.

The nanoemulsion comprises droplets having an average particle size of less than about 3 microns, and the nanoemulsion comprises water, at least one oil, at least one surfactant, and at least one organic solvent. In one embodiment of the invention, the surfactant present in the nanoemulsion is a cationic surfactant. In another embodiment of the invention, the nanoemulsion further comprises a chelating agent. In one embodiment of the invention, nanoemulsions from the present invention, or those derived from the nanoemulsions of the present invention, are diluted. The diluted samples can then be tested to determine if they maintain the desired functionality, such as surfactant concentration, stability, and particle size.

The nanoemulsions can be in the form of any pharmaceutically acceptable dosage form, including but not limited to, liquids, ointments, creams, oils, emulsions, lotions, gels, liquids, bioadhesive gels, sprays, shampoos, aerosols, pastes, foams, sunscreens, capsules, microcapsules, or in the form of an article or carrier, such as a bandage, insert, syringe-like applicator, pessary, powder, talc or other solid, shampoo, cleanser (leave on and wash off product), day creams, night creams, make-up removal creams, foundation creams, make-up removal formulations, protective or skin care body milks, skin care lotions, gels, or foams (such as cleansing or disinfecting lotions), bath compositions, deodorant compositions, aftershave and pre-shave gels or lotions, and agents that favor penetration within the epidermis, the dermis and keratin layers. The nanoemulsion is capable of effectively treating, preventing, and/or minimizing the dermatological conditions described herein, without being systemically absorbed and without significantly irritating the skin.

The nanoemulsions comprise droplets having an average diameter of less than about 3 microns, and the nanoemulsions comprise an aqueous phase, at least one oil, at least one surfactant or detergent, and at least one organic solvent. In one embodiment of the invention, the surfactant present in the nanoemulsion is a cationic surfactant. More than one surfactant or detergent can be present in the nanoemulsions of the invention, and the second surfactant can be the same type (i.e., two cationic surfactants) or the second or third etc. surfactant can be different from the first. For example, the nanoemulsions can comprise a cationic surfactant in combination with a non-ionic surfactant. In another embodiment of the invention, the nanoemulsion further comprises a chelating agent. The organic solvent and the aqueous phase of the invention can be a non-phosphate based solvent.

The nanoemulsions comprise high energy nanometer-sized droplets that permeate into the pilosebaceous unit, dermis and/or epidermis. Droplets having a suitable particle size can permeate skin pores and into the pilosebaceous unit, but can be excluded by tight junctions between epithelial cells and thus do not disrupt tissue matrices or enter blood vessels. This minimizes skin irritation and systemic absorption, but yet provides for a composition which is highly topically bioavailable in the pilosebaceous unit, epidermal and dermal tissues without causing disruption to the normal epithelial matrix.

In one embodiment of the invention, the nanoemulsion comprises: (a) an aqueous phase; (b) about 1% oil to about 80% oil; (c) about 0.1% organic solvent to about 50% organic solvent; (d) about 0.001% surfactant or detergent to about 10% surfactant or detergent; (e) about 0.0005% to about 1.0% of a chelating agent; or (f) any combination thereof. In another embodiment of the invention, the nanoemulsion comprises: (a) about 10% oil to about 80% oil; (b) about 1% organic solvent to about 50% organic solvent; (c) at least one non-ionic surfactant present in an amount of about 0.1% to about 10%; (d) at least one cationic agent present in an amount of about 0.01% to about 2%; (e) about 0.0005% to about 1.0% of a chelating agent; or (f) any combination thereof.

In yet another embodiment of the invention, the nanoemulsion additionally includes at least one suitable or desirable active agent (e.g., a drug or cosmetic active agent). The active agent can be present in a therapeutically effective amount, such as from about 0.001% up to about 99%, about 0.01% up to about 95%, about 0.1% up to about 90%, about 3% up to about 80%, about 5% up to about 60%, about 10% up to about 50%, or any combination thereof (e.g., about 3% up to about 10%).

The quantities of each component present in the nanoemulsion refer to a therapeutic nanoemulsion, and not to a nanoemulsion to be tested in vitro. This is significant, as nanoemulsions tested in vitro, such as the nanoemulsions described in the examples, generally have lower concentrations of oil, organic solvent, surfactant or detergent, and (if present) chelating agent than that present in a nanoemulsion intended for therapeutic use, e.g., topical use. This is because in vitro microbiology studies do not require the nanoemulsion droplets to traverse the skin or other barriers. For topical use, the concentrations of the components must be higher to result in therapeutic levels of nanoemulsion. However, the relative quantities of each component used in a nanoemulsion tested in vitro are applicable to a nanoemulsion to be used therapeutically and, therefore, in vitro quantities can be scaled up to prepare a therapeutic composition, and in vitro data may well be predictive of topical application success.

### A. DEFINITIONS

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “active agent” is used herein to refer to a chemical material or compound that induces a desired beneficial effect when administered topically or subcutaneously, and includes agents that are therapeutically and/or prophylactically effective as pharmaceuticals (“pharmacologically active agents”), as well as agents that are cosmeceutically effective (“cosmeceutically active agents”). Also included are derivatives and analogs of those compounds or classes of compounds specifically mentioned that also induce the desired effect. By an “effective” amount of an active agent is meant a nontoxic but sufficient amount of an active agent to provide the desired beneficial effect. More specifically, by a “therapeutically effective,” “prophylactically effective,” or “cosmeceutically effective” amount is meant a nontoxic but sufficient amount of a beneficial agent to provide the desired therapeutic, prophylactic, or cosmeceutical effect.

The term “aging-related skin condition” relates to any skin condition or disorder associated with, caused by, or affected by, intrinsic aging and/or extrinsic aging. Aging-related skin conditions that may be treated using the present methods and formulations include, but are not limited to, wrinkles, age spots, sun damage (particularly UV radiation-induced oxidative stress), blemishes, hyperpigmented skin, age spots, increased skin thickness, loss of skin elasticity and collagen content, dry skin, lentigines, melasmas, as well as scars.

The terms “buffer” or “buffering agents” refer to materials which when added to a solution, cause the solution to resist changes in pH.

“Carriers” or “vehicles” as used herein refer to carrier materials suitable for incorporation in a topically or subcutaneously applied composition. Carriers and vehicles useful herein include any such materials known in the art, which are nontoxic and do not interact with other components of the formulation in which it is contained in a deleterious manner.

The terms “chelator” or “chelating agent” refer to any materials having more than one atom with a lone pair of electrons that are available to bond to a metal ion.

By “cosmeceutically effective” is meant a nontoxic agent that has medicinal or drug-like properties which, when applied to the surface of skin, beneficially affects the biological functioning of that skin.

The terms “cosmeceutically active agent” and “cosmeceutically active base” are used interchangeably herein to refer to a cosmeceutically effective basic compound or composition of matter which, when topically administered to a human patient, is effective to treat one or more aging-related skin conditions as defined above. Also included are derivatives and analogs of those compounds or classes of compounds specifically mentioned that also induce the desired effect, i.e., treatment of an aging-related skin condition.

By “cosmeceutically acceptable,” such as in the recitation of a “cosmeceutically acceptable carrier,” or a “cosmeceutically acceptable derivative,” is meant a compound that is not biologically or otherwise undesirable, i.e., the compound may be incorporated into a cosmeceutical formulation of the invention and topically administered to a patient without causing any undesirable biological effects or interacting in a deleterious manner with any of the other components of the cosmeceutical formulation in which it is contained. The term “pharmaceutically acceptable” is used in an analogous manner, to refer to a compound or composition that may be incorporated into a pharmaceutical formulation herein (i.e., a cosmeceutical formulation containing one or more pharmacologically active agents) without causing undesirable biological effects or unwanted interaction with other components of the formulation.

The term “dilution” refers to dilution of the nanoemulsions of the present invention or those derived from the nanoemulsions of the present invention using, for example, an aqueous system comprised of PBS or water (such as diH₂O), or other water soluble components, to the desired final concentration.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The droplets have an average diameter of less than about 3 microns.

The terms “pharmaceutically acceptable” or “pharmacologically acceptable,” as used herein, refer to compositions that do not substantially produce adverse allergic or immunological reactions when administered to a host (e.g., an animal or a human). Such formulations include any pharmaceutically acceptable dosage form. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The term “stable” when referring to a “stable nanoemulsion” means that the nanoemulsion retains its structure as an emulsion. A desired nanoemulsion structure, for example, may be characterized by a desired size range, macroscopic observations of emulsion science (is there one or more layers visible, is there visible precipitate), pH, and a stable concentration of one or more the components.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group.

As used herein, the term “topically” refers to application of the compositions of the present invention to the surface of the skin and tissues.

The terms “treating” and “treatment” as used herein refer to reduction in severity and/or elimination of skin related conditions resulting from intrinsic and/or extrinsic aging processes of the skin, or other trauma to the skin resulting in, e.g., a scar. The present method of “treating” a skin condition related to aging, as the term is used herein, refers to the prevention of aging-related skin conditions as well as the treatment of aging-related skin conditions in affected individuals.

### B. VISCOSITY

Examples 6 and 7 below demonstrate that increasing the viscosity of the nanoemulsion can enhance distribution and permeation of the nanoemulsion into the skin, thereby producing a nanoemulsion more effective in an anti-aging and/or anti-wrinkle treatment.

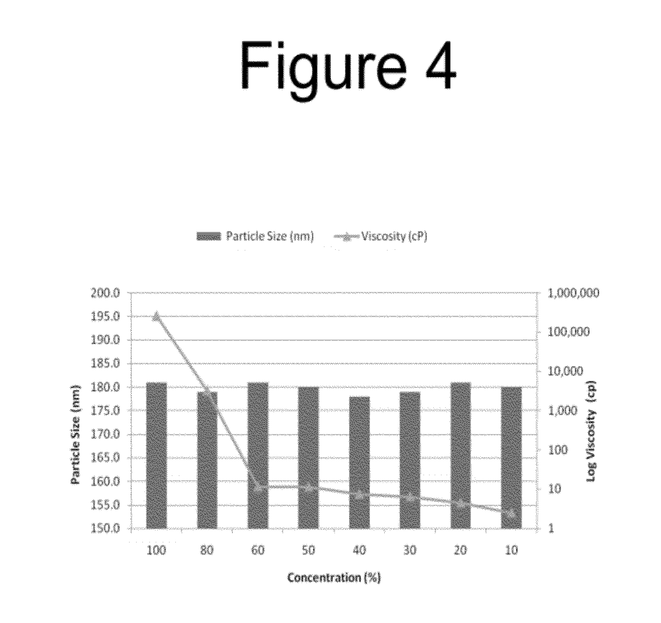

FIG. 4 shows the relationship between the particle size (nm), concentration of active (%), and viscosity of a nanoemulsion. The particle size does not change upon dilution of a nanoemulsion; however viscosity significantly decreases as a function of the decrease in particle concentrations. Thus, embodiment of the invention encompass using dilutions of a nanoemulsion. Table 6 (below) shows the effect dilution of a nanoemulsion has on the concentration of the active (CPC), viscosity, and particle size.

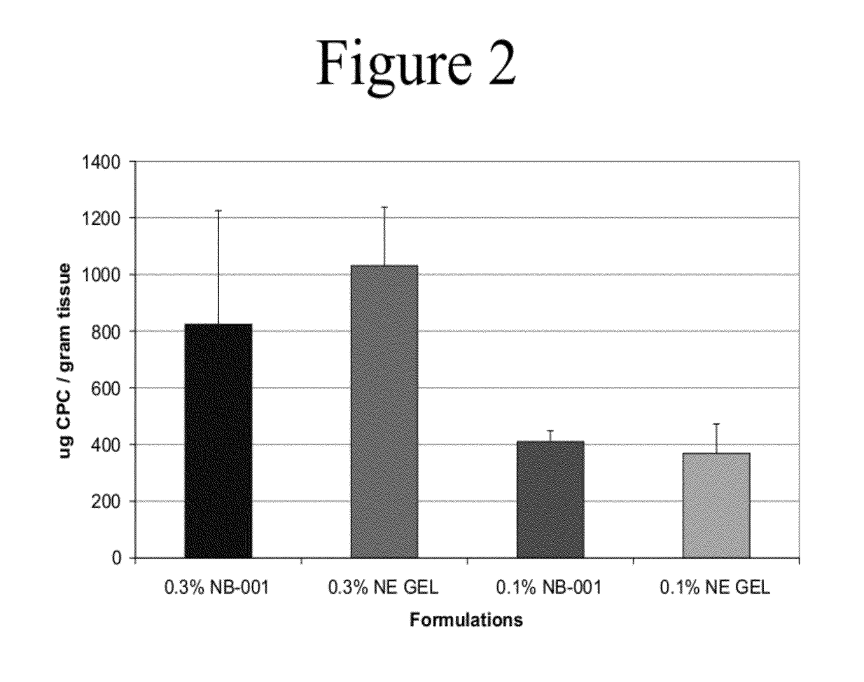

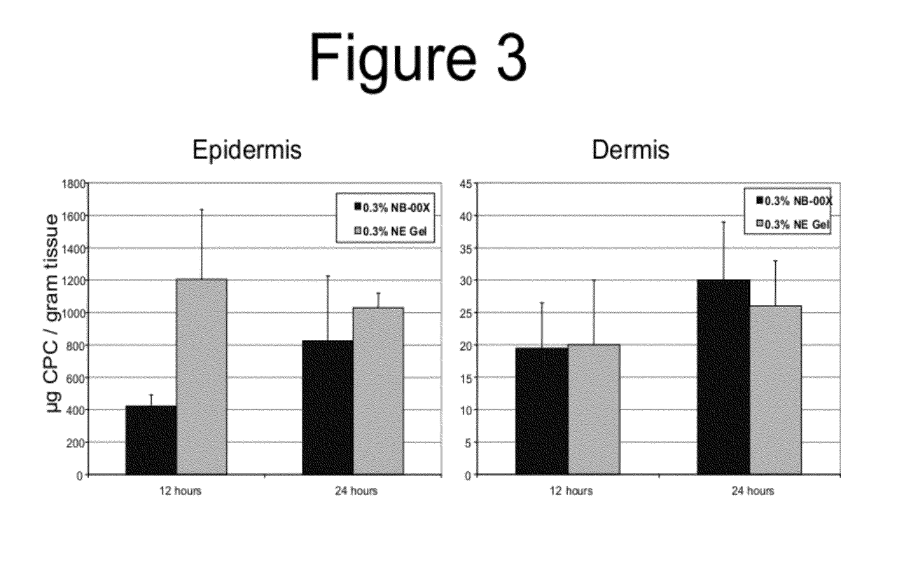

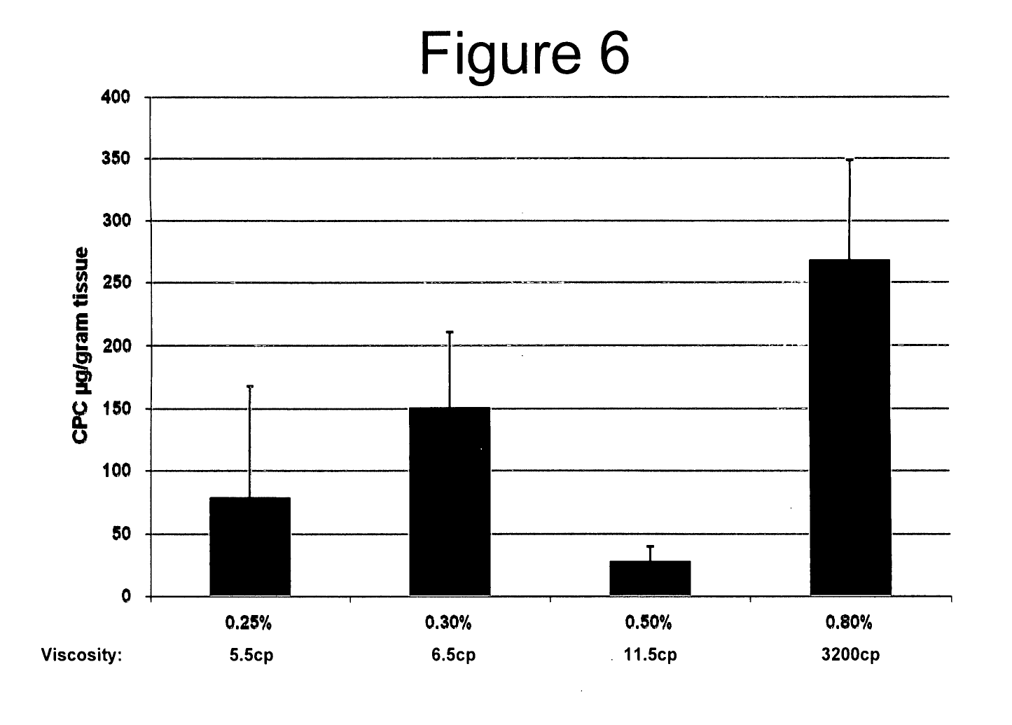

FIGS. 2, 3, 5 and 6 show the results for epidermis and dermis permeation, respectively. Higher viscosity nanoemulsions were found to increase the permeation of the nanoemulsion into the epidermis (FIGS. 2, 3 and FIG. 5) and dermis (FIGS. 3 and 6).

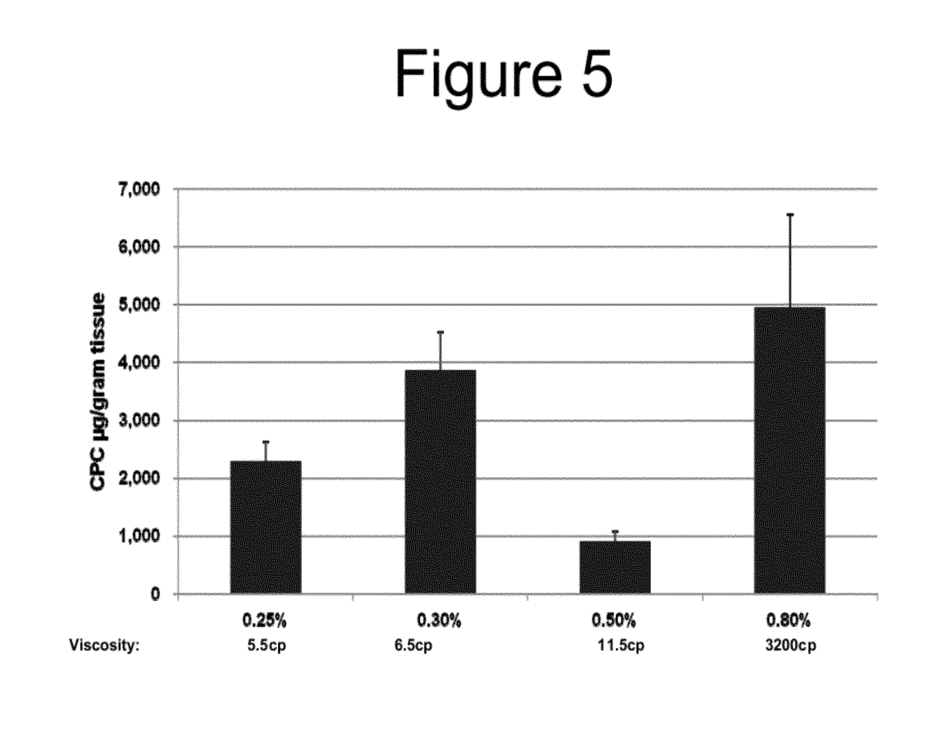

More particularly, as shown in FIGS. 5 and 6, lower concentration nanoemulsions, e.g., 0.25% to 0.30%, are effective in penetrating the skin. Slightly higher or lower concentrations are also effective. However, at a concentration of 0.5%, permeation significantly declined. Surprisingly, higher concentrations such as 0.8% or more showed a dramatic increase in permeation due to the increased viscosity of the composition. It is theorized that the increase in viscosity inhibits or limits the evaporation of water from the skin after application of the emulsion, thus preventing the crystallization of the active from the nanoemulsion and extending the time of permeation of the nanoemulsions into the various skin strata. As an alternative to increasing the concentration of the nanoemulsion, the viscosity of the nanoemulsion can be increased to provide improved therapeutic effectiveness. Examples of methods of increasing the viscosity of a nanoemulsion according to the invention including increasing the concentration of the nanoemulsion (e.g., increasing CPC concentration or the number of nanoemulsion droplets), or adding a thickening agent or gelling agent to the formulation (see e.g., FIGS. 2 and 3).

Thus, in one embodiment of the invention, the nanoemulsion has a viscosity of greater than about 12 centipoise (cP), greater than about 15 cP, greater than about 20 cP, greater than about 25 cP, greater than about 30 cP, greater than about 35 cP, greater than about 40 cP, greater than about 45 cP, greater than about 50 cP, greater than about 55 cP, greater than about 60 cP, greater than about 65 cP, greater than about 70 cP, greater than about 75 cP, greater than about 80 cP, greater than about 85 cP, greater than about 90 cP, greater than about 95 cP, greater than about 100 cP, greater than about 150 cP, greater than about 200 cP, greater than about 300 cP, greater than about 400 cP, greater than about 500 cP, greater than about 600 cP, greater than about 700 cP, greater than about 800 cP, greater than about 900 cP, greater than about 1000 cP, greater than about 1500 cP, greater than about 2000 cP, greater than about 2500 cP, greater than about 3000 cP, greater than about 3500 cP, greater than about 4000 cP, greater than about 4500 cP, greater than about 5000 cP, greater than about 5500 cP, greater than about 6000 cP, greater than about 7000 cP, greater than about 8000 cP, greater than about 9000 cP, greater than about 10,000 cP, greater than about 15,000 cP, greater than about 20,000 cP, greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 150,000 cP, greater than about 200,000 cP, greater than about 250,000 cP, or up to about 259,300 cP.

### C. TEMPERATURE

One tactic that can increase the effectiveness of a nanoemulsion according to the invention in anti-aging and anti-wrinkle treatments is ensuring that the nanoemulsion is at room temperature or warmer prior to application. Thus, in another embodiment of the invention, encompassed are compositions for and methods of treating dermatological conditions described herein, such as anti-aging and anti-wrinkle treatments, comprising application of a nanoemulsion according to the invention, wherein the nanoemulsion is at room temperature (e.g., 20 to 25° C.). In another embodiment of the invention, encompassed are compositions for and methods of treating dermatological conditions described herein, such as anti-aging and anti-wrinkle treatments, comprising application of a nanoemulsion according to the invention, wherein the nanoemulsion has been warmed prior to application. For example, the nanoemulsion can be warmed prior to application to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, about 37° C. or warmer,

### D. STABILITY OF THE NANOEMULSIONS OF THE INVENTION

The nanoemulsions of the invention are stable at about 40° C. and about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the nanoemulsions of the invention are stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsions of the invention are stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### E. NANOEMULSIONS

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion of the present invention comprises droplets having an average diameter size of less than about 3 microns, less than about 2500 nm, less than about 2000 nm, less than about 1500 nm, less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 225 nm, less than about 220 nm, less than about 210 nm, less than about 205 nm, less than about 200 nm, less than about 190 nm, less than about 175 nm, less than about 150 nm, or any combination thereof. In one embodiment, the droplets have an average diameter size greater than about 125 nm and at least 400 nm. In another embodiment, the droplets have an average diameter of 180 nm.

1. Aqueous Phase

The aqueous phase can comprise any type of aqueous phase including, but not limited to, water (e.g., H₂O, distilled water, tap water) and solutions (e.g., phosphate-buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate-buffered saline (PBS). The aqueous phase may further be sterile and pyrogen free.

2. Organic Solvents

Organic solvents in the nanoemulsions of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

3. Oil Phase

The oil in the nanoemulsion of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof. In one aspect of the invention, the volatile oil in the silicone component is different than the oil in the oil phase.

4. Surfactants/Detergent

The surfactant or detergent in the nanoemulsion of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N—N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quaternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, minimum 98% (TLC), CHAPS, for electrophoresis, minimum 98% (TLC), CHAPSO, minimum 98%, CHAPSO, CHAPSO, for electrophoresis, 3-(Decyldimethylammonio)propanesulfonate inner salt, 3-Dodecyldimethylammonio)propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion comprises a cationic surfactant, which can be cetylpyridinium chloride. In other embodiments of the invention, the nanoemulsion comprises a cationic surfactant, and the concentration of the cationic surfactant is less than about 5.0% and greater than about 0.001%. In yet another embodiment of the invention, the nanoemulsion comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion is less than about 5.0% and greater than about 0.001%.

In another embodiment of the invention, the nanoemulsion comprises at least one cationic surfactant and at least one non-cationic surfactant. The non-cationic surfactant is a nonionic surfactant, such as a polysorbate (Tween), such as polysorbate 80 or polysorbate 20.

In one embodiment, the non-ionic surfactant is present in a concentration of about 0.05% to about 7.0%, or the non-ionic surfactant is present in a concentration of about 0.3% to about 4%. In yet another embodiment of the invention, the nanoemulsion comprises a cationic surfactant present in a concentration of about 0.01% to about 2%, in combination with a nonionic surfactant.

5. Active Agents

In a further embodiment, the composition additionally comprises one or more active agents. The presence of such an active agent is not required, and substantial and unexpected anti-aging and/or anti-wrinkle properties are observed without the presence of such an active agent. Thus, the presence of such active agents is to merely enhance the anti-aging and anti-wrinkle properties of the compositions of the invention. In another embodiment, the composition further comprises at least one active agent for rendering the composition suitable as a cosmetic preparation.

In one embodiment, active agents useful in the nanoemulsion compositions of the invention are compounds that provide benefits to the skin and/or provide desirable properties to a composition formulated as a cosmetic or medicinal preparation. The active agent useful in the nanoemulsion compositions of the invention can be a drug substance or a non-drug substance. Examples of non-drug active agents include, but are not limited to, skin lightening agents, tanning agents, skin conditioning agents, skin protectants, emollients and humectants, and sunscreen actives.

Examples of exemplary active agents useful in the nanoemulsion compositions of the invention, such as an active drug substance or an active cosmetic substance, include, but are not limited to, Botulinum toxin type A (Botox®), a retinoid (e.g., vitamin A derivatives, retinol, retinal, tretinoin (retinoic acid, Renova®, Retin-A®), isotretinoin, alitretinoin, etretinate, acitretin, tazarotene (Avage®, Tazorac®), bexarotene and Adapalene), alpha hydroxy acids, beta hydroxy acids, poly hydroxy acids, hydroxyl acids, kinetin, coenzyme Q10, copper peptides, tea extracts (e.g., green, black and oolong tea extracts), antioxidants (e.g., ascorbic acid (vitamin C), glutathione, melatonin, tocopherols, α-tocopherol, tocotrienols (vitamin E), lipoic acid, uric acid, carotenes, ubiquinone (coenzyme Q), thioredoxin, Polyphenolic antioxidants (resveratrol, flavonoids), and carotenoids), colloidal oatmeal (Aveeno®), soybean extract, or any mixture thereof.

In another embodiment, the composition of the invention comprises an anti-viral agent. Such a composition is useful, for example, in treating a herpes virus outbreak following laser treatment, such as ablative laser resurfacing.

In yet another embodiment, the composition of the invention comprises an anti-acne agent. Exemplary topical anti-acne agents include, but are not limited to, benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, adapalene, tretinoin, erythromycin, clindamycin, azelaic acid, hydrocortisone, sodium hyaluronate, sulfur, urea, meclocycline, dapsone, retinoids and retinoid derivatives. Other anti-acne ingredients include Ascorbyl Tetraisopalmitate, Dipotassium Glycyrrhizinate, Ascorbyl Tetraisopalmitate, Niacinamide, alpha bisabolol can also be included in the nanoemulsion of this invention. All of these skin care ingredients have properties that help to reduce and control acne, and acne related problems such as sebum production.

Additional anti-acne agents include acne herbal medicines, such as Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil.

Representative sunscreen drugs are active ingredients that absorb, reflect, or scatter radiation in the UV range at wavelengths from 290 to 400 nanometers. Specific examples include benzophenone-3 (oxybenzone), benzophenone-4 (sulisobenzone), benzophenone-8(dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)]aminobenzoate), ethylhexyl dimethyl PABA (Padimate O), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate (trolamine salicylate), titanium dioxide, and zinc oxide. One skilled in the art will appreciate that other sunscreen agents may be used in the compositions and preparations of the present invention.

Representative skin lightening agents include, but are not limited to, ascorbic acid and derivatives thereof, kojic acid and derivatives thereof, hydroquinone and derivatives thereof, azelaic acid, various plant extracts such as those from licorice, grape seed and bear berry, and mixtures of any one or more of the foregoing. Those skilled in the art will appreciate that other skin lightening agents may be included in the compositions used for some of the methods of the present invention.

As noted above, the compositions may further comprise skin conditioning agents. Such agents comprise substances that enhance the appearance of dry or damaged skin, as well as materials that adhere to the skin to reduce flaking, restore suppleness, and generally improve the appearance of skin. Representative examples of skin conditioning agents include acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and derivatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples of skin conditioning agents include, but are not limited to, lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanbl, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. Skin conditioning agents, other than those listed above, may also be used, as is readily appreciated by those skilled in the art.

In other embodiments, the compositions may include a skin protectant, defined herein as a compound that protects injured or exposed skin or mucous membrane surfaces from harmful or irritating external compounds. Representative examples thereof include algae extract, allantoin, aluminum hydroxide, aluminum sulfate, betaine, camellia sinensis leaf extract, cerebrosides, dimethicone, glucuronolactone, glycerin, kaolin, lanolin, malt extract, mineral oil, petrolatum, potassium gluconate, and talc. Those skilled in the art will readily appreciate that skin protectants, other than those listed above, may be included in the compositions used for the methods of the present invention.

An emollient, as the term is used herein, is a cosmetic ingredient that can help the skin maintain a soft, smooth, and pliable appearance. Emollients are able to provide these benefits, largely owing to their ability to remain on the skin surface or in the stratum corneum to act as a lubricant and reduce flaking. Some examples of emollients, suitable for embodiments of this invention, are acetyl arginine, acetylated lanolin, algae extract, apricot kernel oil PEG-6 esters, avocado oil PEG-11 esters, bis-PEG-4 dimethicone, butoxyethyl stearate, C₁₈C₃₆ acid glycol ester, C₁₂C₁₃ alkyl lactate, caprylyl glycol, cetyl esters, cetyl laurate, coconut oil PEG-10 esters, di-C₁₂C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2acetate, lauryl polyglyceryl-6cetearyl glycol ether, methyl gluceth-20 benzoate, mineral oil, myreth-3 palmitate, octyidecanol, octyldodecanol, odontella aurita oil, 2-oleamido-1,3 octadecanediol, palm glycerides, PEG avocado glycerides, PEG castor oil, PEG-22/dodecyl glycol copolymer, PEG shorea butter glycerides, phytol, raffinose, stearyl citrate, sunflower seed oil glycerides, and tocopheryl glucoside. Those skilled in the art will readily appreciate that emollients, other than those listed above, may also be used.

Humectants are cosmetic ingredients that help maintain moisture levels in skin. Some examples of suitable humectants are: acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea. Other humectants may be used for yet additional embodiments of this invention, as will be appreciated by those skilled in the art.

Examples of antiviral agents include, but are not limited to, nucleoside analogs (e.g., acyclovir (Zovirax®), famciclovir (Famvir®), and valaciclovir (Valtrex®)), amantadine (Symmetrel®), oseltamivir (Tamiflu®), rimantidine (Flumadine®), and zanamivir (Relenza®), Cidofovir (Vistide®), foscarnet (Foscavir®), ganciclovir (Cytovene®), ribavirin (Virazole®), penciclovir (Denavir®), buciclovir, acyclic guanosine derivatives, (E)-5-(2-bromovinyl)-2′-deoxyuridine and structurally related analogues thereof [i.e., the cytosine derivative (E)-5-(2-bromovinyl)-2′-deoxycytidine and the 4′-thio derivative (E)-5-(2-bromovinyl)-2′-deoxy-4′-thiouridine], Nucleoside/Nucleotide Analogues (e.g., Abacavir (Ziagen, ABC), Didanosine (Videx, ddI), Emtricitabine (Emtriva, FTC), Lamivudine (Epivir, 3TC), Stavudine (Zerit, d4T), Tenofovir (Viread, TDF), Zalcitabine (Hivid, ddC), and Zidovudine (Retrovir, AZT, ZDV)); Nonnucleoside Reverse Transcriptase Inhibitors (e.g., Delavirdine (Rescriptor, DLV), Efavirenz (Sustiva, Stocrin, EFV), Etravirine (Intelence, TMC 125), Nevirapine (Viramune, NVP)); Protease Inhibitors (Amprenavir (Agenerase, APV), Atazanavir (Reyataz, ATV), Darunavir (Prezista, DRV, TMC 114), Fosamprenavir (Lexiva, Telzir, FPV), Indinavir (Crixivan, IDV), Lopinavir/Ritonavir (Kaletra), Nelfinavir (Viracept, NFV), Ritonavir (Norvir, RTV), Saquinavir (Invirase, SQV), and Tipranavir (Aptivus, TPV)); Fusion Inhibitors (e.g., Enfuvirtide (Fuzeon, ENF, T-20)); Chemokine Coreceptor Antagonists (e.g., Maraviroc (Selzentry, Celsentri, MVC)); and Integrase Inhibitors (e.g., Raltegravir (Isentress, RAL)). Preferred antiviral agents for incorporation into a nanoemulsion include, but are not limited to, acyclovir (Zovirax®), penciclovir (Demavor®), famciclovir (Famvir®), and valaciclovir (Valtrex®). Other exemplary active agents which can be incorporated into a nanoemulsion for treating a herpes viral outbreak include, but are not limited to, docosanol (Abreva®)

6. Additional Ingredients

Additional compounds suitable for use in the nanoemulsions of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the nanoemulsions of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof.

The nanoemulsion may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion can comprise a chelating agent. In one embodiment of the invention, the chelating agent is present in an amount of about 0.0005% to about 1.0%. Examples of chelating agents include, but are not limited to, ethylenediamine, ethylenediaminetetraacetic acid (EDTA), and dimercaprol, and a preferred chelating agent is ethylenediaminetetraacetic acid.

The nanoemulsion can comprise a buffering agent, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, ˜5 M in H₂O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H₂O, Ammonium formate, ≧99.0% (calc. based on dry substance, NT), Ammonium oxalate monohydrate, ≧99.5% (RT), Ammonium phosphate dibasic solution, 2.5 M in H₂O, Ammonium phosphate dibasic, ≧99.0% (T), Ammonium phosphate monobasic solution, 2.5 M in H₂O, Ammonium phosphate monobasic, ≧99.5% (T), Ammonium sodium phosphate dibasic tetrahydrate, ≧99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, ≧0.1 M Na₂₆CO₃, ≧0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H₂O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H₂O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H₂O, MOPS, ≧99.5% (T), MOPS, for luminescence, ≧99.5% (T), MOPS, for molecular biology, ≧99.5% (T), Magnesium acetate solution, for molecular biology, ˜1 M in H₂O, Magnesium acetate tetrahydrate, ≧99.0% (KT), Magnesium citrate tribasic nonahydrate, ≧98.0% (calc. based on dry substance, KT), Magnesium formate solution, 0.5 M in H₂O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H₂O, Potassium formate solution, 14 M in H₂O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H₂O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, ˜3 M in H₂O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H₂O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H₂O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H₂O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H₂O (colorless solution at 20° C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate—EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≧99.0% (NT), Trizma® base, ≧99.8% (T), Trizma® base, ≧99.8% (T), Trizma® base, for luminescence, ≧99.8% (T), Trizma® base, for molecular biology, ≧99.8% (T), Trizma® carbonate, ≧98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≧99.0% (AT), Trizma® hydrochloride, for luminescence, ≧99.0% (AT), Trizma® hydrochloride, for molecular biology, ≧99.0% (AT), and Trizma® maleate, ≧99.5% (NT).

The nanoemulsion can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsions that may readily be diluted with water to a desired concentration without impairing their anti-fungal or antiyeast properties.

In addition to the foregoing active agents, the compositions employed in the methods of the present invention may also comprise inert and physiologically acceptable carriers or diluents. Suitable carriers or diluents include, but are not limited to water, physiological saline, bacteriostatic saline (e.g., saline containing 0.9 mg/ml benzyl alcohol), petrolatum based creams (e.g., USP hydrophilic ointments and similar creams), various types of pharmaceutically acceptable gels, and short chain alcohols and glycols (e.g., ethyl alcohol and propylene glycol).

In other further embodiments, the compositions employed may comprise additional ingredients such as fatty alcohols, fatty acids, organic or inorganic bases, preserving agents (such as phenoxyethanol and mixtures of various parabens), wax esters, steroid alcohols, triglyceride esters, phospholipids such as lecithin and cephalin, polyhydric alcohol esters, fatty alcohol ethers, hydrophilic lanolin derivatives, hydrophilic beeswax derivatives, cocoa butter waxes, silicon oils, pH balancers, cellulose derivatives, hydrocarbon oils such as palm oil, coconut oil, and mineral oil, and mixtures thereof.

Additional ingredients may be included in the above compositions to vary the texture, viscosity, color and/or appearance thereof, as is appreciated by one of ordinary skill in the art. Accordingly, in a further embodiment, the compositions, in addition to at least one peptide manganese complex, comprise an emulsifying agent, a surfactant, a thickening agent, an excipient or a mixture thereof.

More specifically, emulsifiers and surfactants may be included in those compositions used for the present invention that are formulated as emulsions. Either water-in-oil or oil-in-water emulsions may be formulated. Examples of suitable surfactants and emulsifying agents include nonionic ethoxylated and nonethoxylated surfactants, abietic acid, almond oil PEG, beeswax, butylglucoside caprate, C₁₈C₃₆ acid glycol ester, C₉C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂C₁₃ pareth-3 sulfate, tri-C₁₂C₁₅ pareth-6 phosphate, and trideceths. Other surfactants and emulsifiers may be used, as will be appreciated by those skilled in the art.

Examples of thickening (i.e., viscosity increasing) agents include, but are not limited to, those agents commonly used in skin care preparations, such as acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxytheylcellulose, hydroxypropylcellulose, hydroxpropyl starch, magnesium alginate, methylcellulose, microcrystalline cellulose, pectin, various PEG's, polyacrylic acid, polymethacrylic acid, polyvinyl alcohol, various PPG's, sodium acrylates copolymer, sodium carrageenan, xanthan gum, and yeast beta-glucan. Thickening agents other than those listed above may also be used in related embodiments of the present invention.

### F. PHARMACEUTICAL COMPOSITIONS

The nanoemulsions of the invention may be formulated into pharmaceutical compositions that comprise the nanoemulsion in a therapeutically effective amount and suitable, pharmaceutically-acceptable excipients for topical administration to a human subject in need thereof. Such excipients are well known in the art. By the phrase “therapeutically effective amount” it is meant any amount of the nanoemulsion that is effective in preventing and/or treating acne. One possible way to treat acne is by killing or inhibiting the growth of P. acnes, causing P. acnes to lose pathogenicity, or any combination thereof.

Topical administration includes administration to the skin, including surface of the hair follicle and pilosebaceous unit.

Pharmaceutically acceptable dosage forms for topical administration include, but are not limited to, ointments, creams, liquids, emulsions, lotions, gels, bioadhesive gels, aerosols, pastes, foams, sunscreens, or in the form of an article or carrier, such as a bandage, insert, syringe-like applicator, pessary, powder, talc or other solid, cleanser (leave on and wash off product), and agents that favor penetration within the pilosebaceous gland.

The pharmaceutical compositions may be formulated for immediate release, sustained release, controlled release, delayed release, or any combinations thereof, into the epidermis or dermis, with no systemic absorption. In some embodiments, the formulations may comprise a penetration-enhancing agent for enhancing penetration of the nanoemulsion through the stratum corneum and into the epidermis or dermis. Suitable penetration-enhancing agents include, but are not limited to, alcohols such as ethanol, triglycerides and aloe compositions. The amount of the penetration-enhancing agent may comprise from about 0.5% to about 40% by weight of the formulation.

In some embodiments, the formulation for delivery via a “patch” comprising a therapeutically effective amount of the nanoemulsion is envisioned. As used herein a “patch” comprises at least a topical formulation and a covering layer, such that the patch can be placed over the area to be treated. Preferably, the patch is designed to maximize delivery through the stratum corneum and into the epidermis or dermis, while minimizing absorption into the circulatory system, and little to no skin irritation, reducing lag time, promoting uniform absorption, and reducing mechanical rub-off and dehydration.

Adhesives for use with the drug-in-adhesive type patches are well known in the art. Suitable adhesive include, but are not limited to, polyisobutylenes, silicones, and acrylics. These adhesives can function under a wide range of conditions, such as, high and low humidity, bathing, sweating etc. Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; or and hydrogels (e.g., high molecular weight polyvinylpyrrolidone and oligomeric polyethylene oxide). The most preferred adhesive is a pressure sensitive acrylic adhesive, for example Durotak® adhesives (e.g., Durotak® 2052, National Starch and Chemicals). The adhesive may contain a thickener, such as a silica thickener (e.g., Aerosil, Degussa, Ridgefield Park, N.J.) or a crosslinker such as aluminumacetylacetonate.

Suitable release liners include but are not limited to occlusive, opaque, or clear polyester films with a thin coating of pressure sensitive release liner (e.g., silicone-fluorsilicone, and perfluorcarbon based polymers.

Backing films may be occlusive or permeable and are derived from synthetic polymers like polyolefin oils polyester, polyethylene, polyvinylidine chloride, and polyurethane or from natural materials like cotton, wool, etc. Occlusive backing films, such as synthetic polyesters, result in hydration of the outer layers of the stratum corneum while non-occlusive backings allow the area to breath (i.e., promote water vapor transmission from the skin surface). More preferably the backing film is an occlusive polyolefin foil (Alevo, Dreieich, Germany). The polyolefin foil is preferably about 0.6 to about 1 mm thick.

The shape of the patch can be flat or three-dimensional, round, oval, square, and have concave or convex outer shapes, or the patch or bandage can also be segmented by the user into corresponding shapes with or without additional auxiliary means.

The nanoemulsions of the invention can be applied and/or delivered utilizing electrophoretic delivery/electrophoresis. Such transdermal methods, which comprise applying an electrical current, are well known in the art.

The pharmaceutical compositions for administration may be applied in a single administration or in multiple administrations. The pharmaceutical compositions are topically or subcutaneously applied for at least once a week, at least twice a week, at least once a day, at least twice a day, multiple times daily, multiple times weekly, biweekly, at least once a month, or any combination thereof. The pharmaceutical compositions are topically applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years. Between applications, the application area may be washed to remove any residual nanoemulsion.

Preferably, the pharmaceutical compositions are applied to the skin area in an amount of from about 0.001 mL/cm² to about 5.0 mL/cm². An exemplary application amount and area is about 0.2 mL/cm², although any amount from 0.001 mL/cm² up to about 5.0 mL/cm² can be applied. Following topical administration, the nanoemulsion may be occluded or semi-occluded. Occlusion or semi-occlusion may be performed by overlaying a bandage, polyoleofin film, impermeable barrier, or semi-impermeable barrier to the topical preparation. Preferably, after application, the treated area is covered with a dressing. Typically, for a method of the present invention, aside from the content of the composition used, a small amount of the composition (from about 1 ml to about 5 ml) is applied to exposed areas of skin from a suitable container or applicator, and, if necessary, the composition is then spread over and/or rubbed into the skin using the hand, finger, or other suitable device. Each composition disclosed herein is typically packaged in a container that is appropriate in view of the composition's viscosity and intended use by the consumer. For example, a lotion or fluid cream may be packaged in a bottle, roll-ball applicator, capsule, propellant-driven aerosol device, or a container fitted with a manually operated pump. A cream may simply be stored in a non-deformable bottle, or in a squeeze container, such as a tube or a lidded jar.

### G. EXEMPLARY NANOEMULSIONS

Several exemplary nanoemulsions are described below, although the methods of the invention are not limited to the use of such nanoemulsions. The components and quantity of each can be varied as described herein in the preparation of other nanoemulsions. Unless otherwise noted, all concentrations are expressed in terms of % w/w.

TABLE 1
Exemplary Therapeutically Effective Nanoemulsions

| Form. | Soy- | | Etha- | | | |
|---|---|---|---|---|---|---|
| (CPC | bean | Tween | nol | CPC % | EDTA | H₂O |
| % w/v) | oil % | 20 % | % | (mg/mL) | % (mM) | % |
| Formulation | 31.4 | 2.96 | 3.37 | 0.53 (5) | 0.037 (1) | 61.70 |
| #1; 0.50% | | | | | | |
| Formulation | 15.7 | 1.48 | 1.68 | 0.27 (2.5) | 0.0185 (0.5) | 80.85 |
| #2; 0.25% | | | | | | |
| Formulation | 62.79 | 5.92 | 6.73 | 1.068 (10) | 0.075 (2) | 23.42 |
| #3; 1.0% | | | | | | |
| Formulation | 18.84 | 1.78 | 2.02 | 0.320 (3) | 0.0224 (0.6) | 77.03 |
| #4; 0.3% | | | | | | |
| Formulation | 6.28 | 0.59 | 0.67 | 0.107 (1) | 0.0075 (0.2) | 92.34 |
| #5; 0.1% | | | | | | |

### H. METHODS OF MANUFACTURE

The nanoemulsions of the invention can be formed using classic emulsion forming techniques. See e.g., U.S. 2004/0043041. See also the method of manufacturing nanoemulsions described in U.S. Pat. Nos. 6,559,189, 6,506,803, 6,635,676, 6,015,832, and U.S. Patent Publication Nos. 20040043041, 20050208083, 20060251684, and 20070036831, and WO 05/030172, all of which are specifically incorporated by reference. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water. The nanoemulsions of the invention are stable, and do not decompose even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures/consistencies ranging from that of a semi-solid cream to that of a thin lotion and can be applied topically by hand and sprayed onto a surface. As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in an emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use.

The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles (e.g., pressurized spray bottles).

### I. EXAMPLES

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### Example 1

### Preparation of Nanoemulsions

Exemplary emulsions are produced by mixing a water-immiscible oil phase into an aqueous phase with a proprietary manufacturing method. The two phases (aqueous phase and oil phase) are combined together and processed to yield an emulsion. The emulsion is further processed to achieve the desired particle size. For the gel formulation, a thickening agent, such as Klucel can be added to the nanoemulsion. For example, Klucel is dissolved in water or any aqueous solvent and added to the nanoemulsion to achieve the desired concentration.

Exemplary nanoemulsions (“NB-00X”) shown in Table 1 comprised, in an aqueous medium, soybean oil, Tween 20® as a nonionic surfactant, ethanol, cetylpyridinium chloride (CPC) as a cationic surfactant, EDTA, and water, and optionally, a thickening agent for the gel formulation.

TABLE 2
Compositions of the Nanoemulsions (NB-00X) and Nanoemulsion Gels (NB-Gel).
The percentages are wt/wt, unless otherwise noted.

| | Soybean oil | Tween | Ethanol | CPC | EDTA | Klucel | Water |
|---|---|---|---|---|---|---|---|
| Formulation | % | 20 % | % | % (w/v) | % | % | % |
| 0.1% NB-00X | 6.279 | 0.592 | 0.679 | 0.107 | 0.0074 | 0 | 92.34 |
| 0.3% NB-00X | 18.837 | 1.776 | 2.037 | 0.320 | 0.022 | 0 | 77.01 |
| 0.1% NB-Gel | 6.279 | 0.592 | 20.679 | 0.107 | 0.0074 | 1% | 92.34 |
| 0.3% NB-Gel | 18.837 | 1.776 | 22.037 | 0.320 | 0.022 | 1% | 77.01 |

The nanoemulsions were then formulated at 10 different concentrations, as two-fold serial dilutions from 0.0064% NB-00X (equivalent to 64 μg CPC/ml) to 0.0000125% NB-00X (equivalent to 0.125 μg CPC/ml). Each dilution contained varying concentrations of soybean oil, Tween 20®, ethanol, CPC, and EDTA. Combination products were also prepared; stock emulsions containing NB-00X gel (3 mg CPC/ml)+2% salicyclic acid or NB-00X gel+0.5% benzoyl peroxide (BPO) were serially diluted two-fold.

A. Source of Drugs

NB-00X (liquid formulation), lot X1151 and NB-00X gel, were prepared at concentrations of 6000 μg/ml and 3000 μg/ml respectively. These lots were prepared at NanoBio Corporation from NB-PO-004-FP manufactured at Contract Pharmaceutical Laboratories (CPL), Buffalo, N.Y., USA. Placebo lots (placebo for NB-00X gel, contains thickening agent and additional solvent) were prepared from manufactured at NanoBio Corporation. Since nanoemulsions are not a single small molecule, their relative activity can be expressed in terms of the concentration of cationic surfactant present. Thus, the activity of NB-00X formulations is expressed in microgram CPC per ml. NB-00X gel (lot X1158) contained a thickening agent in addition to the components of NB-00X. Combination products were made as stock emulsions containing NB-00X gel (3 mg CPC/ml)+2% salicyclic acid or NB-00X gel+0.5% benzoyl peroxide (BPO).

Salicylic acid was purchased from J. T. Baker as VWR International catalog number 0300-01. BPO in the form of Invisible Acne cream containing 10% BPO was purchased from Meijer Distribution Inc. (Grand Rapids, Mich.).

Since NB-00X is a nanoemulsion and is preferentially taken up by the transfollicular route (Ciotti et al., “Novel nanoemulsion NB-001 permeates skin by the follicular route,” Abstr. 45^{th} Intersci. Conf., Antimicrob. Agents Chemother., abstr. A-1898 (2008)), incorporation of a drug, such as an anti-acne drug, into the nanodroplets could be used to effectively deliver these additional agents to the desired site.

### Example 2

### Skin Permeation Studies

The purpose of this example was to evaluate the in vitro absorption into the epidermis and dermis of nanoemulsions according to the invention. Pig skin was used as an animal model.

A. In Vitro Skin Model

The in vitro skin model has proven to be a valuable tool for the study of percutaneous absorption of topically applied compounds. The model uses excised skin mounted in specially designed diffusion chambers that allow the skin to be maintained at a temperature and humidity that match typical in vivo conditions. Franz, T J, “Percutaneous absorption: on the relevance of in vitro data,” J. Invest. Dermatol., 64:190-195 (1975). A finite dose of formulation is applied to the epidermis, and outer surface of the skin and compound absorption is measured by monitoring its rate of appearance in the receptor solution bathing the dermal surface of the skin. Data defining total absorption, rate of absorption, as well as skin content can be accurately determined in this model. The method has historic precedent for accurately predicting in vivo percutaneous absorption kinetics. Franz T J, “The finite dose technique as a valid in vitro model for the study of percutaneous absorption in man,” In: Skin: Drug Application and Evaluation of Environmental Hazards, Current Problems in Dermatology, vol. 7, Simon et al. (Eds) (Basel, Switzerland, S. Karger, 1978, pp 58-68.)

B. Nanoemulsions Used in the Study

TABLE 3

| | Soybean oil | Tween | Ethanol | CPC % | EDTA | Klucel | Water |
|---|---|---|---|---|---|---|---|
| Formulation | % | 20 % | % | (w/v) | % | % | % |
| 0.1% NB-001 | 6.279 | 0.592 | 0.679 | 0.107 | 0.0074 | 0 | 92.34 |
| 0.3% NB-001 | 18.837 | 1.776 | 2.037 | 0.320 | 0.022 | 0 | 77.01 |
| 0.1% NB-Gel | 6.279 | 0.592 | 20.679 | 0.107 | 0.0074 | 1% | 92.34 |
| 0.3% NB-Gel | 18.837 | 1.776 | 22.037 | 0.320 | 0.022 | 1% | 77.01 |

C. Pig Skin

Full thickness, abdominal skin (˜1000 μm thickness) from 5.4 month old male Hanford swine (S/N 5353) was used in permeation studies and obtained from Sinclair Research Center, Inc, Auxvasse, Mo. The subcutaneous fat was removed using a scalpel and the skin was stored in aluminum foil pouches at ˜70° C. until use. At time of use, the skin was thawed by placing the sealed pouch in 30° C. water for approximately five minutes. Thawed skin was removed from the pouch and cut into circular discs (30 mm diameter) to fit between the donor and receiver sides of the permeation chambers.

D. Franz Diffusion Cell Methodology: Conditions, Parameters, Procedure

Percutaneous absorption was measured using the in vitro cadaver skin finite dose technique. Thirty mm of swine skin was placed onto the surface of each cell. Each receptor compartment was filled with distilled water, pH 7 and the donor compartment was left open to ambient laboratory conditions. The receptor compartment spout was covered with a screw cap to minimize evaporation of the receptor solution. All cells were mounted in a diffusion apparatus in which the receptor solution was maintained at 37° C. The receptor compartment was maintained at 34.5° C. in a water bath and was stirred with a magnetic stirrer.

The skin was equilibrated before applying 113 μL of each test article onto the skin surface.

E. Sampling (Receptor Sampling, Epidermis, Dermis, Surface Swabs)

Twenty-four hours after application of the first dose, the surface of the dosing area was rinsed with ethanol solution and swabbed independently to remove all residual formulation from the skin surface. Receptor solution was also sampled at 24 hours from the receptor of each cell and filtered into vials.

Skin samples were collected as described above; weights of the epidermal and dermal samples were obtained. The epidermal and dermal tissues were extracted with absolute ethanol, sonicated, and filtered and assayed using HPLC.

F. Epidermal and Dermal Calculations

The amount of CPC that permeated into the epidermis, dermis and the receptor compartment was determined by HPLC. A standard concentration of CPC was generated and used to determine the concentration of CPC in the dosing area. The levels of CPC in each skin area are represented as the amount per wet tissue weight (μg/grams)±the standard deviation.

The results of CPC permeation studies are shown in FIGS. 2 and 3. There was an increase in the delivery of the CPC marker to the epidermis and dermis with the 0.3% NB-00X as compared to the 0.1% NB-001X formulation, as expected. The gels for the 0.1% NB-00X and 0.3% NB-00X did not hinder delivery. The amount of CPC found in the receptor compartment at 24 hours was below the level of detection (5 μg/ml) for all the formulations.

At the twelve hour time point, the gel formulation delivered two-fold higher levels of CPC into the epidermis, indicating a fast rate of delivery. The dermal levels were similar (See FIG. 3).

### Example 3

### Skin Permeation Studies With Nanoemulsion+Second Active Ingredient

The purpose of this example was to evaluate the in vitro absorption of a second active ingredient from the nanoemulsion into the epidermis and dermis according to the invention. Two different active ingredients were evaluated following incorporation into the nanoemulsion: benzoyl peroxide or adapalene.

The nanoemulsion test formulations contained 0.3% (w/w) NB-001 with 0.1% (w/w) Adapalene or 0.3% (w/w) NB-00X with either 0.5% (w/w) BPO or 2.5% (w/w) BPO. Control formulations tested were commercially available products; Persa-Gel-10 (10% benzoyl peroxide; Johnson & Johnson), 0.1% Differin Lotion (0.1% Adapelene; Galderma) and 0.3% Differin Gel (0.3% Adapalene gel; Galderma).

A. In Vitro Skin Model

The in vitro skin model has proven to be a valuable tool for the study of percutaneous absorption of topically applied compounds. The model uses excised skin mounted in specially designed diffusion chambers that allow the skin to be maintained at a temperature and humidity that match typical in vivo conditions. Franz, T J, “Percutaneous absorption: on the relevance of in vitro data,” J. Invest. Dermatol., 64:190-195 (1975). A finite dose of formulation is applied to the epidermis and outer surface of the skin and compound absorption is measured by monitoring its rate of appearance in the receptor solution bathing the dermal surface of the skin. Data defining total absorption, rate of absorption, as well as skin content can be accurately determined in this model. The method has historic precedent for accurately predicting in vivo percutaneous absorption kinetics. Franz T J, “The finite dose technique as a valid in vitro model for the study of percutaneous absorption in man,” In: Skin: Drug Application and Evaluation of Environmental Hazards, Current Problems in Dermatology, vol. 7, Simon et al. (Eds) (Basel, Switzerland, S. Karger, 1978, pp 58-68.)

B. Nanoemulsions Used in the Study

TABLE 4
Quantitative Composition of 0.3% NB-00X with Benzoyl Peroxide

| | 0.3% NB-00X with | 0.3% NB-00X with |
|---|---|---|
| | 0.5% Benzoyl | 2.5% Benzoyl |
| Components | Peroxide (% w/w) | Peroxide (% w/w) |
| Cetylpyridinium Chloride | 0.3204 | 0.3204 |
| Monohydrate, USP | | |
| Purified Water, USP | qs | qs |
| Soybean Oil, USP | 18.84 | 18.84 |
| Dehydrated Alcohol, USP | 2.019 | 2.019 |
| (anhydrous ethanol) | | |
| Polysorbate 20, NF | 1.776 | 1.776 |
| Edetate Disodium Dihydrate, | 0.0222 | 0.0222 |
| (EDTA) USP | | |
| persa-gel ®-10^{b} | 5 | 25 |
| (contains 10% benzoyl | | |
| peroxide) | | |
| Sterile Water for Irrigation | 45 | 25 |
| USP | | |
| Total (%) | 100 | 100 |

^{b}contains 10% benzoyl peroxide and other excipients (carbomer, disodium EDTA, hydroxypropyl-methylcellulose, laureth 4, sodium hydroxide, water)

TABLE 5
Quantitative Composition of 0.3% NB-001 with 1% Adapalene

| | 0.3% NB-003 with 1% |
|---|---|
| Components | Adapalene (% w/w) |
| Cetylpyridinium Chloride | 0.3204 |
| Monohydrate, USP | |
| Purified Water, USP | 76.9224 |
| Soybean Oil, USP | 18.84 |
| Dehydrated Alcohol, USP | 2.019 |
| (anhydrous ethanol) | |
| Polysorbate 20, NF | 1.776 |
| Edetate Disodium Dihydrate, | 0.0222 |
| (EDTA) USP | |
| Adapalene | 0.1 |
| Total (%) | 100 |

C. Pig Skin

Full thickness, abdominal skin (˜1000 μm thickness) from 5.4 month old male Hanford swine (S/N 5353) was used in permeation studies and obtained from Sinclair Research Center, Inc, Auxvasse, Mo. The subcutaneous fat was removed using a scalpel and the skin was stored in aluminum foil pouches at −70° C. until use. At time of use, the skin was thawed by placing the sealed pouch in 30° C. water for approximately five minutes. Thawed skin was removed from the pouch and cut into circular discs (30 mm diameter) to fit between the donor and receiver sides of the permeation chambers.

D. Human Cadaver Skin

Cryopreserved, dermatomed human cadaver abdominal skin from a Caucasian female donor was used in permeation studies and obtained from Life Legacy tissue organ donor bank. Cadaver skin was stored in aluminum foil pouches at −70° C. until use. At time of use, the skin was thawed by placing the sealed pouch in 37° C. water for approximately five minutes. Thawed skin was removed from the pouch and cut into circular discs (30 mm diameter) to fit between the donor and receiver sides of the permeation chambers.

E. Franz Diffusion Cell Methodology: Conditions, Parameters, Procedure

Percutaneous absorption was measured using the in vitro cadaver skin finite dose technique. Thirty mm of swine skin was placed onto the surface of each cell. Each receptor compartment was filled with distilled water, with a solubilizing solvent (such as 10% ethanol) and or an anti-oxidant (0.1% BHT) and the donor compartment was left open to ambient laboratory conditions. The receptor compartment spout was covered with a screw cap to minimize evaporation of the receptor solution. All cells were mounted in a diffusion apparatus in which the receptor solution was maintained at 37° C. The receptor compartment was maintained at 34.5° C. in a water bath and was stirred with a magnetic stirrer. The skin was equilibrated before applying 113 μL of each test article onto the skin surface. The test article was applied twice at eight hour intervals for the swine skin studies and once a day for the human cadaver skin study.

F. Sampling (Receptor Sampling, Epidermis, Dermis, Surface Swabs)

Twenty-four hours after application of the first dose, the surface of the dosing area was rinsed with ethanol solution and swabbed independently to remove all residual formulation from the skin surface. Receptor solution was also sampled at 24 hours from the receptor of each cell and filtered into vials.

Skin samples were collected as described above; and weights of the epidermal and dermal samples were obtained. The epidermal and dermal tissues were extracted with absolute ethanol, sonicated, and filtered and the second active ingredient assayed using HPLC.

G. Epidermal and Dermal Calculations

The amount of the second active ingredient that permeated into the epidermis, dermis and the receptor compartment was determined by HPLC. A standard concentration of the active was generated and used to determine the concentration of active in the dosing area. The levels of active in each skin area are represented as the amount per wet tissue weight (μg/grams)±the standard deviation.

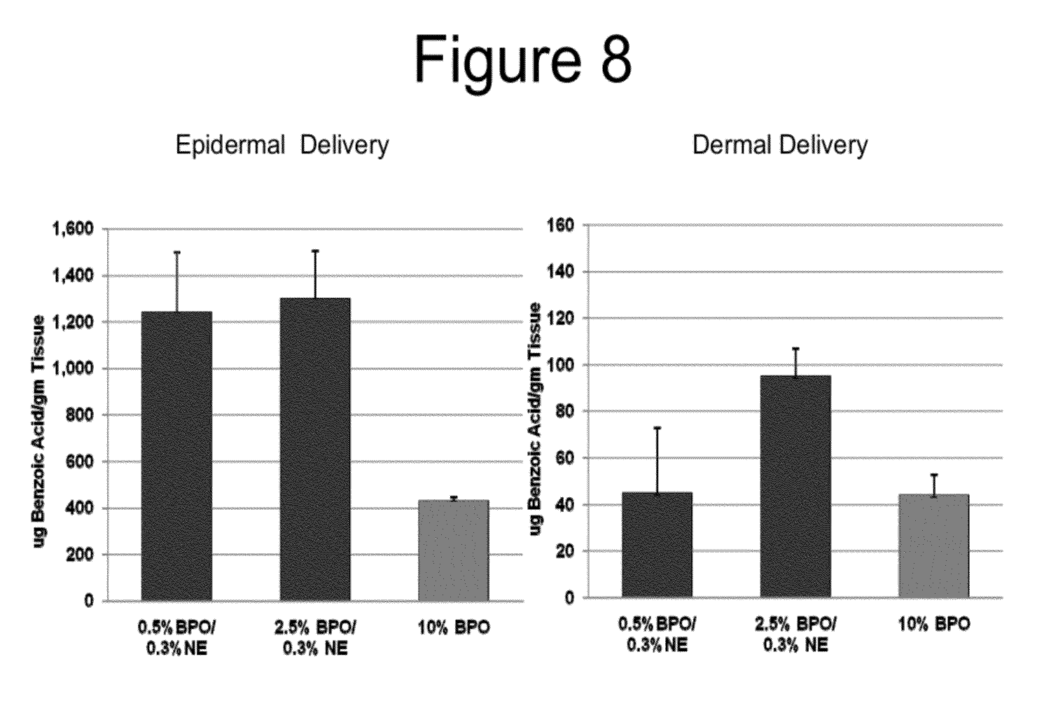

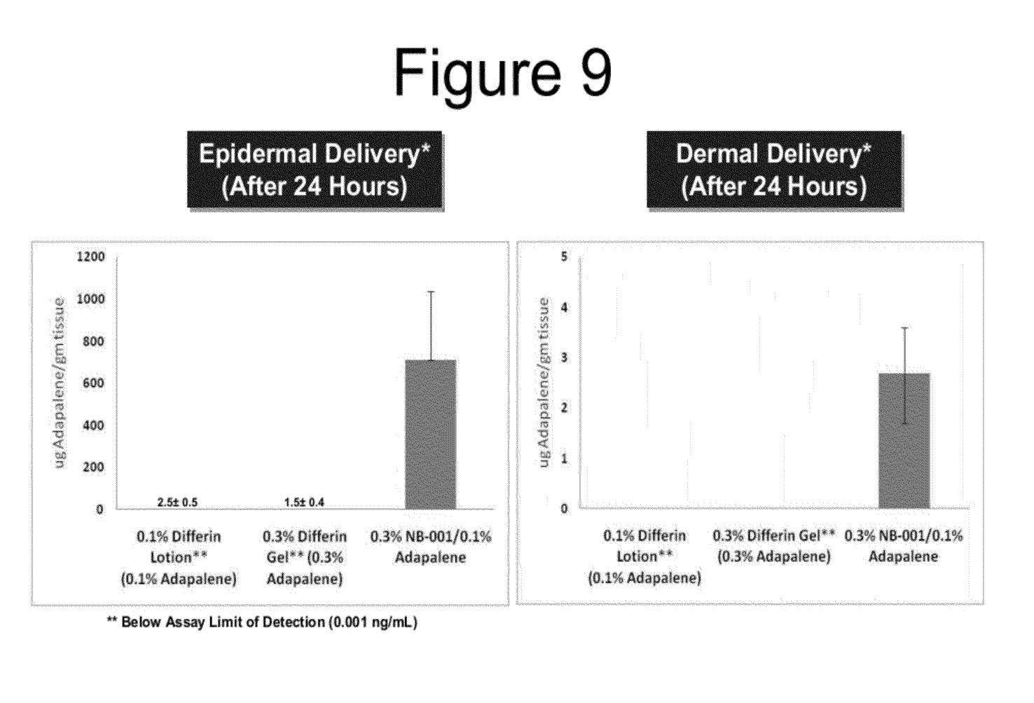

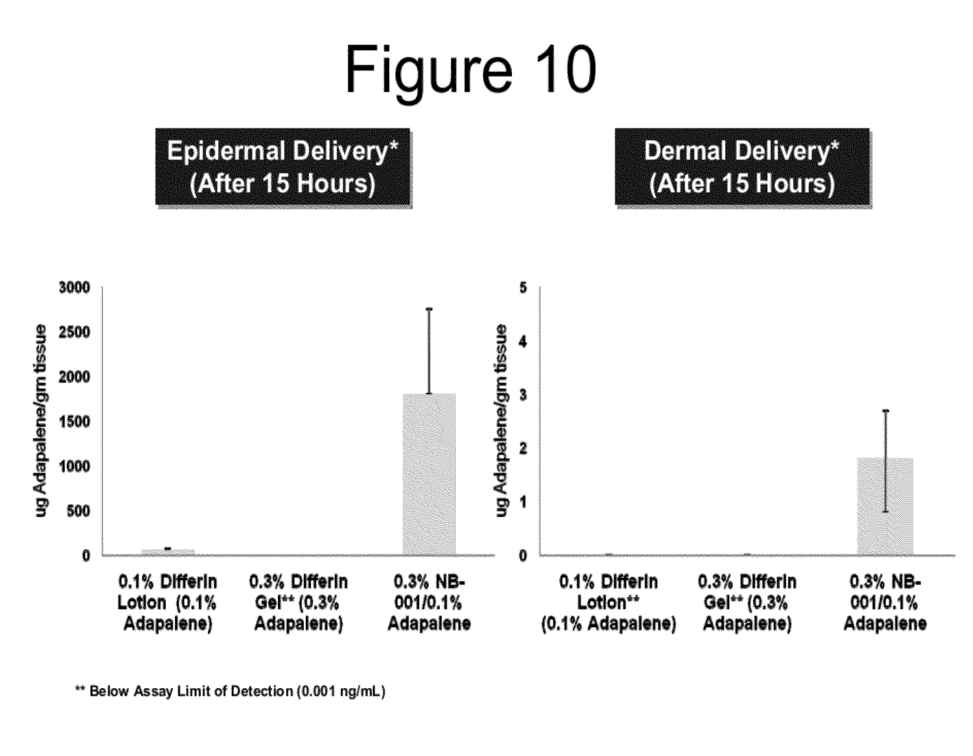

The results of benzoyl peroxide and adapalene permeation studies are shown in FIGS. 8, 9 and 10, respectively. There was an increase in the delivery of the second active incorporated in the nanoemulsion to the epidermis and dermis as compared to a commercial preparation.

### Example 4

### Viscosity

The purpose of this example was to evaluate the effect of concentration of a nanoemulsion has on the viscosity of the nanoemulsion.

FIG. 4 shows the relationship between the particle size (nm), concentration of active (%), and viscosity of a nanoemulsion. The particle size does not change upon dilution of a nanoemulsion; however viscosity significantly decreases as a function of the decrease in particle concentrations. Table 6 shows the effect dilution of a nanoemulsion has on the concentration of the active (CPC), viscosity, and particle size.

TABLE 6
NB-001 Process Optimization - Dilution

| Percentage of | | | |
|---|---|---|---|
| Concentrated | Theoretical CPC | | Particle Size |
| NB-001 | Potency (% wt/v) | Viscosity (cP) | (nm) |
| 100% | 1.0 | 259,300 | 181 |
| 80% | 0.8 | 3200 | 179 |
| 60% | 0.6 | 11.5 | 181 |
| 50% | 0.5 | 11.5 | 180 |
| 40% | 0.4 | 7.5 | 178 |
| 30% | 0.3 | 6.5 | 179 |
| 20% | 0.2 | 4.5 | 181 |
| 10% | 0.1 | 2.5 | 180 |

### Example 5

### Viscosity and Permeation

The purpose of this example was to evaluate the effect viscosity of a nanoemulsion has on the permeation of the active into the dermis and epidermis.

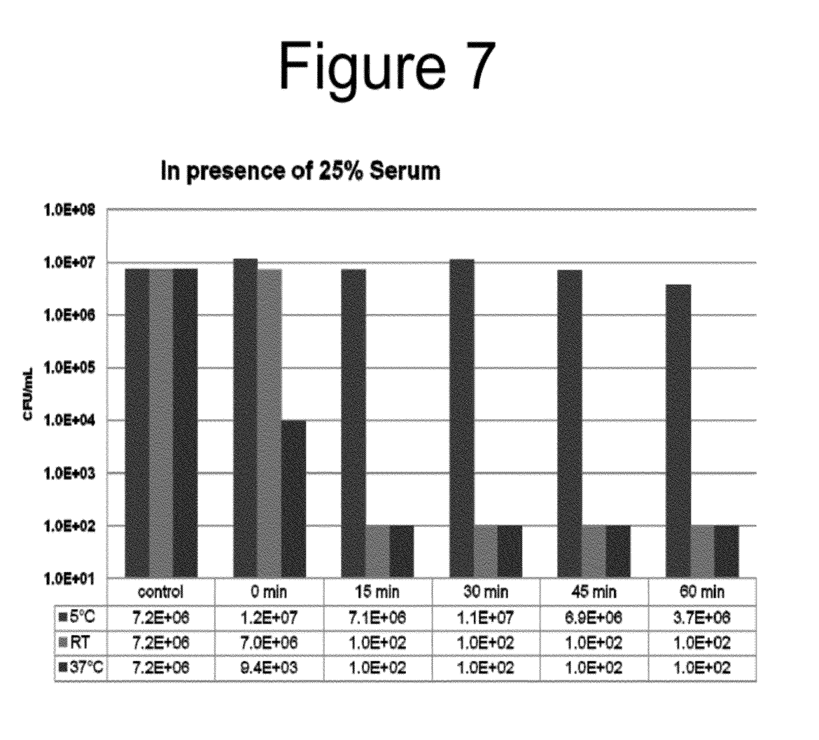

A permeation study was conducted using the protocol described in Example 4 with five skin sections (n=5). Four different concentrations of nanoemulsion (see Table 6) were tested: 0.25%, 0.30%, 0.50% and 0.80%. FIGS. 6 and 7 show the results for epidermis and dermis permeation, respectively. Specifically, FIG. 6 shows the results of the permeation study utilizing pig skin epidermis with 5 skin sections (n=5) following administration of a nanoemulsion (NB-003) twice daily (BID). Higher viscosity (greater than 1000 cps) nanoemulsions (e.g. 0.8% NB-003) were found to have greater permeation of the nanoemulsion into the epidermis.

Similarly, FIG. 7 shows the results of a permeation study utilizing pig skin epidermis with 5 skin sections (n=5) following administration of a nanoemulsion (NB-003) twice daily (BID). Higher viscosity (greater than 1000 cps) nanoemulsions (e.g. 0.8% NB-003) were found to deliver three times the amount of the surfactant, cetylpyridinium chloride (CPC) to the dermis as compared to a lower viscosity nanoemulsion (e.g., 0.25% NB-003).

Thus, increasing the viscosity of a nanoemulsion can increase the permeation of the nanoemulsion into the dermis and epidermis, thereby producing a composition more effective in killing bacteria or other organisms.

### Example 6

The purpose of this example was to evaluate the efficacy of a nanoemulsion in treating the signs of aging present on facial skin.

Two different nanoemulsions were prepared as in Example 1; 0.1% NB-003 and 0.3% NB-003.

TABLE 7

| | Soybean | | | CPC | | |
|---|---|---|---|---|---|---|
| | oil | Tween | Ethanol | % | EDTA | Water |
| Formulation | % | 20 % | % | (w/v) | % | % |
| 0.1% NB-003 | 6.279 | 0.592 | 0.679 | 0.107 | 0.0074 | 92.34 |
| 0.3% NB-003 | 18.837 | 1.776 | 2.037 | 0.320 | 0.022 | 77.01 |

Two groups of human subjects topically applied either 0.1% NB-003 or 0.3% NB-003 to facial skin twice a day for a period of 29 days. 8 subjects were in Group 1 and topically applied the 0.1% NB-003 formulation daily to facial skin, and 20 subjects were in Group 2 and topically applied the 0.3% NB-003 formulation daily to facial skin. At the end of the 29 day test period, the subjects were polled regarding various aspects of the effectiveness of the nanoemulsion in treating and/or minimizing signs of aging present on the skin. The following aspects of the nanoemulsion were evaluated: (1) effectiveness of the nanoemulsion in improving the smoothness and/or softness of skin (i.e., making the skin feel smoother and softer following treatment); (2) effectiveness of the nanoemulsion in improving the overall appearance of skin; (3) effectiveness of the nanoemulsion in evening out skin tone and texture; (4) effectiveness of the nanoemulsion in improving the clarity and/or radiance of skin; (5) effectiveness of the nanoemulsion in making the skin look younger; and (6) effectiveness of the nanoemulsion in making wrinkles appear softer and/or less prominent. The results of the polling questions are shown below in the table below.

The results were significant. In particular, 75% of 0.1% NB-003 users reported that skin was smoother and softer following treatment. Similarly, 37.5% of 0.1% NB-003 users reported that following treatment skin demonstrated more clarity and/or radiance, and that wrinkles appeared softer and/or less prominent. Moreover, 25% of 0.1% NB-003 users reported that following treatment the overall appearance of skin was improved, skin tone and skin texture was more even, and skin looked and/or felt younger.

Significant and unexpected results were also observed with the 0.3% NB-003 group, where following treatment 40% reported that skin was smoother and softer, 35% reported that the overall appearance of skin was improved and that skin demonstrated more clarity and/or radiance, 30% reported that skin tone and skin texture was more even, and 20% reported that skin looked and/or felt younger and that wrinkles appeared softer and/or less prominent.

Collectively, following treatment with either 0.1% NB-003 and 0.3% NB-003, 50% of subjects reported that skin felt smoother and softer, 35.7% reported that skin had more clarity and/or radiance, 32.1% reported that the overall appearance of skin was improved, 28.6% reported that skin tone and texture was more even, 25% reported that wrinkles appeared softer and/or less prominent, and 21.4% reported that skin looked and or felt younger.

These results demonstrate the effectiveness of the nanoemulsions of the invention in anti-aging treatments for skin.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method of treating, preventing, minimizing, and/or diminishing a dermatological condition comprising administering a nanoemulsion to a subject, wherein:
(a) the nanoemulsion comprises droplets having an average diameter of less than about 3 microns; and
(b) the nanoemulsion droplets comprise an oil phase with at least one oil, an aqueous phase comprising at least one surfactant, at least one organic solvent, and water.

2. The method of claim 1, wherein the dermatological condition is selected from the group consisting of (1) fine to moderate wrinkles, (2) liver spots or age spots (lentigines or solar lentigines), (3) uneven skin tone and/or texture, (4) sun-damaged skin or photodamaged skin (particularly UV radiation-induced oxidative stress), (5) blemishes, (6) hyperpigmented skin, (7) increased skin thickness, (8) dry skin, (9) loss of skin elasticity and collagen content, (10) melasmas (atypical pigmentation or hyper-pigmentation of the skin), (11) skin clarity and/or radiance, (12) skin smoothness and/or softness, (13) scars, (14) pore size, (14) hydration, (15) skin smoothness, (16) skin tightness, and any combination thereof.

3. The method of claim 1, wherein the nanoemulsion has a viscosity selected from the group consisting of greater than about 12 centipoise (cP), greater than about 15 cP, greater than about 20 cP, greater than about 25 cP, greater than about 30 cP, greater than about 35 cP, greater than about 40 cP, greater than about 45 cP, greater than about 50 cP, greater than about 55 cP, greater than about 60 cP, greater than about 65 cP, greater than about 70 cP, greater than about 75 cP, greater than about 80 cP, greater than about 85 cP, greater than about 90 cP, greater than about 95 cP, greater than about 100 cP, greater than about 150 cP, greater than about 200 cP, greater than about 300 cP, greater than about 400 cP, greater than about 500 cP, greater than about 600 cP, greater than about 700 cP, greater than about 800 cP, greater than about 900 cP, greater than about 1000 cP, greater than about 1500 cP, greater than about 2000 cP, greater than about 2500 cP, greater than about 3000 cP, greater than about 3500 cP, greater than about 4000 cP, greater than about 4500 cP, greater than about 5000 cP, greater than about 5500 cP, greater than about 6000 cP, greater than about 7000 cP, greater than about 8000 cP, greater than about 9000 cP, greater than about 10,000 cP, greater than about 15,000 cP, greater than about 20,000 cP, greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 150,000 cP, greater than about 200,000 cP, greater than about 250,000 cP, or up to about 259,300 cP.

4. The method of claim 1, wherein:
(a) the nanoemulsion is at room temperature at the time of administration; or
(b) prior to application the nanoemulsion is warmed to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, and about 37° C.

5. The method of claim 1, wherein:
(a) the nanoemulsion droplets have an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 225 nm, less than about 220 nm, less than about 210 nm, less than about 205 nm, less than about 200 nm, less than about 190 nm, less than about 175 nm, less than about 150 nm, less than about 100 nm, greater than about 50 nm, greater than about 70 nm, greater than about 125 nm, and any combination thereof;
(b) the nanoemulsion droplets have an average diameter greater than about 125 nm and less than about 450 nm; or
(c) any combination thereof.

6. The method of claim 1, wherein:
(a) the nanoemulsion is applied topically or subcutaneously;
(b) the nanoemulsion is applied topically to any superficial skin structure;
(c) the nanoemulsion is applied to the facial or neck tissue; or
(d) any combination thereof.

7. The method of claim 1, wherein:
(a) the nanoemulsion is in a dosage form selected from the group consisting of liquids, ointments, creams, oils, emulsions, lotions, gels, liquids, bioadhesive gels, sprays, shampoos, aerosols, pastes, foams, sunscreens, capsules, microcapsules, in the form of an article or carrier, such as a bandage, insert, syringe-like applicator, pessary, powder, talc or other solid, shampoo, cleanser, day creams, night creams, make-up removal creams, foundation creams, make-up removal formulations, protective or skin care body milks, skin care lotions, gels, or foams, bath compositions, deodorant compositions, aftershave and pre-shave gels or lotions;
(b) the nanoemulsion is a controlled release formulation, sustained release formulation, immediate release formulation; or
(c) any combination thereof.

8. The method of claim 1, wherein the nanoemulsion further comprises at least one drug active agent, at least one non-drug active agent, at least one active agent having anti-aging and/or anti-wrinkle properties, at least one active cosmetic substance, at least one antiviral active agent, at least one anti-acne active agent, or any combination thereof.

9. The method of claim 8, wherein the agent is selected from the group consisting of skin lightening agents, tanning agents, skin conditioning agents, skin protectants, sunscreen agent, emollients, humectants, Botulinum toxin type A, a retinoid, vitamin A derivatives, retinol, retinal, tretinoin, retinoic acid, isotretinoin, alitretinoin, etretinate, acitretin, tazarotene, bexarotene, and adapalene, alpha hydroxy acids, beta hydroxy acids, poly hydroxy acids, hydroxyl acids, kinetin, coenzyme Q10, copper peptides, tea extracts, green tea extracts, black tea extracts, oolong tea extracts, antioxidants, ascorbic acid, glutathione, melatonin, tocopherols, α-tocopherol, tocotrienols, vitamin E, lipoic acid, uric acid, carotenes, ubiquinone (coenzyme Q), thioredoxin, Polyphenolic antioxidants, resveratrol, flavonoids, and carotenoids.

10. The method of claim 1, wherein the nanoemulsion comprises:
(a) an aqueous phase;
(b) about 1% oil to about 80% oil;
(c) about 0.1% organic solvent to about 50% organic solvent;
(d) at least one surfactant present in an amount of about 0.001% surfactant to about 10% surfactant; and
(e) about 0.0005% to about 1.0% of a chelating agent.

11. The method of claim 10, wherein the nanoemulsion comprises:
(a) at least one non-ionic surfactant present in an amount of about 0.1% to about 10%; and
(b) at least one cationic agent present in an amount of about 0.01% to about 2%.

12. The method of claim 1, wherein:
(a) the nanoemulsion is stable at about 40° C. and about 75% relative humidity for a time period selected from the group consisting of up to about 1 month, up to about 3 months, up to about 6 months, up to about 12 months, up to about 18 months, up to about 2 years, up to about 2.5 years, and up to about 3 years;
(b) the nanoemulsion is stable at about 25° C. and about 60% relative humidity for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, and up to about 5 years;
(c) the nanoemulsion is stable at about 4° C. for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years; or
(d) any combination thereof.

13. The method of claim 1, wherein the organic solvent:
(a) is selected from the group consisting of C₁-C₁₂ alcohol, diol, triol, dialkyl phosphate, tri-alkyl phosphate, semi-synthetic derivatives thereof, and combinations thereof;
(b) is an alcohol which is selected from the group consisting of a nonpolar solvent, a polar solvent, a protic solvent, and an aprotic solvent;
(c) is selected from the group consisting of ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof; or
(d) any combination thereof.

14. The method of claim 1, wherein the oil:
(a) is any cosmetically or pharmaceutically acceptable oil:
(b) is non-volatile;
(c) is selected from the group consisting of animal oil, vegetable oil, natural oil, synthetic oil, hydrocarbon oils, silicone oils, and semi-synthetic derivatives thereof;
(d) is selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or
(e) any combination thereof.

15. The method of claim 1, wherein the nanoemulsion comprises a volatile oil, wherein:
(a) the volatile oil can be the organic solvent, or the volatile oil can be present in addition to an organic solvent;
(b) the volatile oil is a terpene, monoterpene, sesquiterpene, carminative, azulene, semi-synthetic derivatives thereof, or combinations thereof;
(c) the volatile oil is selected from the group consisting of a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, and combinations thereof or
(d) any combination thereof.

16. The method of claim 1, wherein the surfactant is:
(a) a pharmaceutically acceptable ionic surfactant, a pharmaceutically acceptable nonionic surfactant, a pharmaceutically acceptable cationic surfactant, a pharmaceutically acceptable ionic surfactant, a pharmaceutically acceptable anionic surfactant, or a pharmaceutically acceptable zwitterionic surfactant;
(b) a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant;
(c) a polymeric surfactant which is selected from the group consisting of a graft copolymer of a poly(methyl methacrylate) backbone with at least one polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, and combinations thereof;
(d) selected from the group consisting of ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, and mixtures thereof;
(e) a non-ionic lipid selected from the group consisting of glyceryl laurate, glyceryl myristate, glyceryl dilaurate, glyceryl dimyristate, semi-synthetic derivatives thereof, and mixtures thereof;
(f) a polyoxyethylene fatty ether having a polyoxyethylene head group ranging from about 2 to about 100 groups;
(g) an alkoxylated alcohol having the structure shown in formula I below:
R₅—(OCH₂CH₂)_{y}—OH Formula I
wherein R₅ is a branched or unbranched alkyl group having from about 6 to about 22 carbon atoms and y is between about 4 and about 100, and preferably, between about 10 and about 100;
(h) an alkoxylated alcohol which is an ethoxylated derivative of lanolin alcohol;
(i) is nonionic and is selected from the group consisting of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, Heptaethylene glycol monodecyl ether, Heptaethylene glycol monotetradecyl ether, Heptaethylene glycol monododecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O—(N-heptylcarbamoyl)-alpha-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182 Dibenzoate, semi-synthetic derivatives thereof, and combinations thereof;
(j) the surfactant is cationic and is selected from the group consisting of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof;
(k) the surfactant is anionic and is selected from the group consisting of a carboxylate, a sulphate, a sulphonate, a phosphate, Chenodeoxycholic acid, Chenodeoxycholic acid sodium salt, Cholic acid, ox or sheep bile, Dehydrocholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-dodecanesulfonate, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, Ursodeoxycholic acid, semi-synthetic derivatives thereof, and combinations thereof;
(1) the surfactant is zwitterionic and is selected from the group consisting of an N-alkyl betaine, lauryl amindo propyl dimethyl betaine, an alkyl dimethyl glycinate, an N-alkyl amino propionate, CHAPS, minimum 98%, CHAPS, minimum 98%, CHAPS, for electrophoresis, minimum 98%, CHAPSO, minimum 98%, CHAPSO, CHAPSO, for electrophoresis, 3-(Decyldimethylammonio)propanesulfonate inner salt, 3-(Dodecyldimethylammonio)propanesulfonate inner salt, inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof; or
(m) any combination thereof.

17. The method of claim 1, wherein the nanoemulsion comprises at least one cationic surfactant.

18. The method of claim 17, wherein the nanoemulsion comprises a cationic surfactant, and wherein:
(a) the cationic surfactant is cetylpyridinium chloride;
(b) the concentration of the cationic surfactant is less than about 5.0% and greater than about 0.001%;
(c) the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, and greater than about 0.001%; or
(d) any combination thereof.

19. The method of claim 1, wherein the nanoemulsion comprises at least one cationic surfactant and at least one non-cationic surfactant.

20. The method of claim 19, wherein:
(a) the non-cationic surfactant is a nonionic surfactant;
(b) the non-cationic surfactant is a nonionic surfactant which is a polysorbate;
(c) the non-cationic surfactant is a nonionic surfactant which is polysorbate 20 or polysorbate 80 or polysorbate 60;
(d) the non-cationic surfactant is a nonionic surfactant and the non-ionic surfactant is present in a concentration of about 0.05% to about 7.0%;
(e) the non-cationic surfactant is a nonionic surfactant and the non-ionic surfactant is present in a concentration of about 0.5% to about 4%;
(f) the cationic surfactant is present in a concentration of about 0.5% to about 2%, or
(f) any combination thereof.

21. The method of claim 1, wherein the nanoemulsion further comprises:
(a) at least one preservative;
(b) at least one a pH adjuster;
(c) at least pharmaceutically acceptable buffer;
(d) at least one chelating agent;
(e) at least one silicone component; or
(d) any combination thereof.

22. The method of claim 21, wherein:
(a) the preservative is selected from the group consisting of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic Acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof;
(b) the pH adjuster is selected from the group consisting of diethyanolamine, lactic acid, monoethanolamine, triethylanolamine, sodium hydroxide, sodium phosphate, semi-synthetic derivatives thereof, and combinations thereof;
(c) the buffer is selected from the group consisting of 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, ˜5 M in H₂O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H₂O, Ammonium formate, ≧99.0% (calc. based on dry substance, NT), Ammonium oxalate monohydrate, ≧99.5% (RT), Ammonium phosphate dibasic solution, 2.5 M in H₂O, Ammonium phosphate dibasic, ≧99.0% (T), Ammonium phosphate monobasic solution, 2.5 M in H₂O, Ammonium phosphate monobasic, ≧99.5% (T), Ammonium sodium phosphate dibasic tetrahydrate, ≧99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, ≧0.1 M Na₂CO₃, ≧0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H₂O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H₂O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H₂O, MOPS, ≧99.5% (T), MOPS, for luminescence, ≧99.5% (T), MOPS, for molecular biology, ≧99.5% (T), Magnesium acetate solution, for molecular biology, ˜1 M in H₂O, Magnesium acetate tetrahydrate, ≧99.0% (KT), Magnesium citrate tribasic nonahydrate, ≧98.0% (calc. based on dry substance, KT), Magnesium formate solution, 0.5 M in H₂O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H₂O, Potassium formate solution, 14 M in H₂O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H₂O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, ˜3 M in H₂O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H₂O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H₂O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H₂O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H₂O (colorless solution at 20° C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate—EDTA buffer solution, for molecular biology, TRIS buffered saline, 10× concentrate, TRIS glycine SDS buffer solution, for electrophoresis, 10× concentrate, TRIS phosphate—EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≧99.0% (NT), Trizma® base, ≧99.8% (T), Trizma® base, ≧99.8% (T), Trizma® base, for luminescence, ≧99.8% (T), Trizma® base, for molecular biology, ≧99.8% (T), Trizma® carbonate, ≧98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≧99.0% (AT), Trizma® hydrochloride, for luminescence, ≧99.0% (AT), Trizma® hydrochloride, for molecular biology, ≧99.0% (AT), and Trizma® maleate, ≧99.5% (NT);
(d) the chelating agent (i) is present in an amount of about 0.0005% to about 1.0%: (ii) is selected from the group consisting of ethylenediamine, ethylenediaminetetraacetic acid, and dimercaprol; or (iii) a combination thereof;
(e) the silicone component comprises at least one volatile silicone oil, wherein:
(i) the volatile silicone oil can be the sole oil in the silicone component or it can be combined with other silicone and non-silicone oils, and wherein the other oils can be volatile or non-volatile;
(ii) the volatile oil used in the silicone component is different than the oil in the oil phase;
(iii) the silicone component is selected from the group consisting of methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof; or
(f) any combination thereof.

23. The method of claim 1, wherein the water is present in Phosphate Buffered Saline (PBS).

24. The method of claim 1, wherein the nanoemulsion is topically applied:
(a) in a single administration;
(b) for at least once a week, at least twice a week, at least once a day, at least twice a day, multiple times daily, multiple times weekly, biweekly, at least once a month, or any combination thereof;
(c) for a period of time selected from the group consisting of about one week, about two weeks, about three weeks, about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years;
(d) followed by washing the application area to remove any residual nanoemulsion; or
(f) any combination thereof.

25. The method of claim 1, wherein the nanoemulsion droplets enter the pilosebaeous gland (unit), hair follicle, epidermis, dermis, or a combination thereof.
